(19) United States
(12) Patent Application Publication
(10) Pub. No.: US 2017/0173049 A1
(43) Pub. Date: Jun. 22, 2017

(54) WITHACNISTIN COMPOUNDS FOR TREATMENT OF CANCER

(71) Applicant: UNIVERSITY OF SOUTH FLORIDA, TAMPA, FL (US)

(72) Inventor: SAID M. SEBTI, TAMPA, FL (US)

(21) Appl. No.: 15/367,917

(22) Filed: Dec. 2, 2016

Related U.S. Application Data
(63) Continuation of application No. 11/701,722, filed on Feb. 2, 2007, now abandoned.
(60) Provisional application No. 60/764,936, filed on Feb. 2, 2006.
(60) Provisional application No. 60/781,213, filed on Mar. 10, 2006.

(51) Int. Cl. A61K 31/585

(52) U.S. Cl. CPC A61K 31/585

(57) ABSTRACT

The subject invention pertains to the treatment of tumors and cancerous tissues and the prevention of tumorigenesis and malignant transformation through the modulation of STAT3 intracellular signaling. The subject invention concerns pharmaceutical compositions containing one or more withacnistin compounds, or a pharmaceutically acceptable salt or derivative thereof. In one embodiment, the subject invention concerns a composition comprising a mixture of withacnistin, 3-methoxy-2,3-dihydrowithacnistin, and 3-ethoxy-2,3-dihydrowithacnistin, or a salt or derivative of any of the foregoing. Another aspect of the invention concerns methods of inhibiting the growth of a tumor by administering one or more withacnistin compounds, or a pharmaceutically acceptable salt or derivative thereof, to a patient, wherein the tumor is characterized by the constitutive activation of the STAT3 intracellular signaling pathway. The present invention further pertains to methods of moderating the STAT3 signaling pathway in vitro or in vivo using one or more withacnistin compounds, or a pharmaceutically acceptable salt or derivative thereof.

### CROSS-REFERENCE TO RELATED APPLICATIONS

The present application is a continuation of U.S. patent application Ser. No. 11/701,722, filed Feb. 2, 2007, which claims the benefit of U.S. Provisional Application Ser. No. 60/764,936, filed Feb. 2, 2006, and U.S. Provisional Application Ser. No. 60/781,213, filed Mar. 10, 2006, each of which is hereby incorporated by reference herein in its entirety, including any figures, tables, nucleic acid sequences, amino acid sequences, and drawings.

### BACKGROUND OF INVENTION

Signal transducers and activators of transcription (STATs) are a family of seven proteins (STATs 1, 2, 3, 4, 5a, 5b, and 6) unique in their ability both to transducer extracellular signals and regulate transcription directly. STATs transduce extracellular signals from cytokines such as interleukin-6 and interferons or growth factors such as platelet-derived growth factor (PDGF) and epidermal growth factor (EGF). Upon activation of these receptors, STATs are recruited to the plasma membrane where they become activated via phosphorylation of conserved tyrosine residues either directly by receptor tyrosine kinases, for example, PDGF receptor (PDGFR) and EGF receptor (EGFR) or by nonreceptor tyrosine kinases, for example, Src and JAK. Phosphorylated STAT proteins either homo- or heterodimerize via reciprocal phosphotyrosine-SH2 interactions after which the STAT dimers translocate to the cell nucleus where they bind DNA at STAT-specific binding sites.

In normal cells STAT (signal transducer and activator of transcription) proteins have been identified as important regulators of diverse physiological functions such as immune response, inflammation, proliferation, differentiation, development, cell survival, and apoptosis (Ihle, J. N. and Kerr, I. M. Trends Genet., 1995, 11: 69-74; Schindler, C. and Darnell, J. E., Jr. Annu. Rev. Biochem., 1995, 64: 621-651; Horvath, C. M. and Darnell, J. E. Curr. Opin. Cell Biol., 1997, 9: 233-239; Stark, G. R. et al. Annu. Rev. Biochem., 1998, 67: 227-264). STAT signaling is tightly regulated in normal cells, either through inhibition of upstream signaling proteins (e.g., internalization of receptors) or negative regulators of sarcoma (Src) and Januse kinase (JAK) proteins, such as suppressor of cytokine signaling (SOCS) proteins, and Src family and JAK phosphatases (e.g., CD45 and SHP-2) (Irie-Sasaki, J. et al. Nature, 2001, 409: 349-354; Myers, M. P. et al. J. Biol. Chem., 2001, 276: 47771-47774; Lefebvre, D. C. et al. Biochim. Biophys. Acta, 2003, 1650: 40-49; Lehmann, U. et al. J. Biol. Chem., 2003, 278, 661-671). STAT proteins have been demonstrated to be directly negatively regulated by SOCs proteins, by protein inhibitors of activated STATs (PIAS), by SHP phosphatases, and recent evidence has shown both Grb2 and GRIM-19 to be novel regulators of STAT3 activation (Lufei, C. et al. EMBO J., 2003, 22: 1325-1335; Zhang, T. et al. Biochem. 1, 2003, 376: 457-464; Wormald, S. and Hilton, D. J. J. Biol. Chem., 2004, 279: 821-824). However, in both tumor cells and tissues, disregulation and constitutive activation of STATs, especially STAT3 and STAT5, have been demonstrated to be important to the proliferation and antiapoptotic activity of tumor cells (Bowman, T. and Jove, R. Cancer Control, 1999, 6: 615-619; Turkson, J. and Jove, R. Oncogene, 2000, 19: 6613-6626).

STATs have been shown to play active roles at all levels of tumorigenesis. STATs are responsible for generating proproliferative signals (e.g., Cyclin D1, survivin; Sinibaldi, D. et al. Oncogene, 2000, 19: 5419-5427; Aoki, Y. et al. Blood, 2003, 101: 1535-1542) and have been shown to upregulate antiapoptotic proteins (e.g., Bcl-XL, Bcl-2; Catlett-Falcone, R. et al. Immunity, 1999, 10: 105-115). In addition, STAT3 has been demonstrated to upregulate VEGF expression, which is necessary for angiogenesis and the maintenance of tumor vasculature (Niu, G. et al. Oncogene, 2002, 21: 2000-2008). Finally, STAT3 has been implicated in the inhibition of immune responses to tumor growth by blocking expression of proinflammatory factors (Wang, T. et al. Nat. Med., 2004, 10: 48-54). Unregulated activation of STAT3 and STAT5 has been demonstrated in a variety of tumor types, including breast carcinoma, prostate cancer, melanoma, multiple myeloma, and leukemia among others (Shuai, K. et al. Oncogene, 1996, 13: 247-254; Garcia, R. et al. Oncogene, 2001, 20: 2499-2513; Garcia, R. et al. Cell Growth Differ., 1997, 8: 1267-1276; Catlett-Falcone, R. et al. Immunity, 1999, 10: 105-115; Mora, L. B. et al. Cancer Res., 2002, 62: 6659-6666; Niu, G. et al. Oncogene, 2002, 21:7001-7010). Various genetic alterations can lead to constitutive activation of either STAT3 or STAT5 (e.g., overexpression of EGFR and ErbB2; Fernandes, A. et al. Int. J. Cancer, 1999, 83: 564-570; Berclaz, G. et al. Int. J. Oncol., 2001, 19: 1155-1160). Autocrine and paracrine production of IL-6 results in activation of STAT3 in prostate cancer and multiple myeloma (Catlett-Falcone, R. et al. Immunity, 1999, 10: 105-115; Mora, L. B. et al. Cancer Res., 2002, 62: 6659-6666), while the oncogene BCR-Abl has been demonstrated to act through constitutive tyrosine phosphorylation of STAT5 in chronic myelogenous leukemia (Shuai, K. et al. Oncogene, 1996, 13: 247-254). Various other tyrosine kinases, for example, TEL-JAK2, v-Src, and c-Kit, may require activation of downstream signaling pathways including STAT3 and STAT5 (Yu, C. L. et al. Science, 1995, 269: 81-83; Cao, X. et al. Mol. Cell. Biol., 1996, 16: 1595-1603; Ning, Z. Q. et al. Blood, 2001, 97: 3559-3567; Spiekermann, K. et al. Exp. Hematol., 2002, 30: 262-271; Paner, G. P. et al. Anticancer Res., 2003, 23: 2253-2260).

On the basis of the importance of STAT3 in tumor progression and survival, researchers have begun to focus on STAT3 as a viable molecular target for cancer chemotherapeutics (Turkson, J. and Jove, R. Oncogene, 2000, 19: 6613-6626). Several different approaches can be taken for the inhibition of the STAT signaling pathway: targeting receptor-ligand interactions; inhibition of upstream STAT-activating receptor tyrosine kinases and nonreceptor tyrosine kinases; activation of STAT phosphatases and other negative regulators of STATs; and inhibition of STAT dimerization, nuclear translocation, DNA binding, or DNA transcription. Studies with antisense, gene therapy, and RNA interference (siRNA) (Niu, G. et al. Cancer Res., 1999, 59: 5059-5063; Niu, G. et al. Oncogene, 2002, 21: 2000-2008; Konnikova, L. et al. BMC Cancer, 2003, 3: 23) have demonstrated that inhibition of STAT3 signaling suppresses tumor growth and induces apoptosis in cell lines and mouse models, validating STAT3 as a target for molecular intervention. Recently, pharmacological approaches to STAT inhibition have resulted in the identification of peptides capable of blocking STAT dimerization (Turkson, J. et al. J. Biol. Chem., 2001, 276: 45443-45455; Turkson, J. et al. Mol. Cancer Ther., 2004, 3: 261-269) and identification of the natural product curcumin as an inhibitor of the IL-6/JAK/STAT signaling pathway (Bharti, A. C. et al. J. Immunol., 2003, 171: 3863-3871). The present inventor has identified the natural product, cucurbitacin I (JSI-124) as a dual inhibitor of phospho-JAK2 and phospho-STAT3 levels in cancer cells (Blaskovich, M. A. et al. Cancer Res., 2003, 63: 1270-1279).

### BRIEF SUMMARY OF THE INVENTION

The subject invention concerns the treatment of tumors and cancerous tissues and the prevention of tumorigenesis and malignant transformation through the disruption of STAT3 intracellular signaling.

The experimental data described in U.S. Patent Application Publication No. 20040138189 and Sun et al. (“Cucurbitacin Q: a selective STAT3 activation inhibitor with potent antitumor activity”, Oncogene, 2005, 24: 3236-3245) that were obtained using the compound identified as “cucurbitacin Q” (Cuc Q) were actually obtained from a mixture of withacnistin, 3-methoxy-2,3-dihydrowithacnistin, and 3-ethoxy-2,3-dihydrowithacnistin. Several years ago, the National Cancer Institute (NCI) accepted samples from a submitter and entered them into their inventory system using the names and structures provided by the submitter without independently verifying, at that time, the chemical identity of the samples. NCI provided one of the samples, which was designated by NCI identifier NSC 135075 and represented to be cucurbitacin Q, to the present inventor, who then characterized its anti-cancer properties. Subsequently, upon chemical analysis, NCI determined that NSC 135075 had been misidentified as Cuc Q. NSC 135075 is actually a mixture of withacnistin, 3-methoxy-2,3-dihydrowithacnistin, and 3-ethoxy-2,3-dihydrowithacnistin, with withacnistin being the major constituent of the mixture (see nuclear magnetic resonance spectra of FIGS. 6 and 5B, and mass spectrum of FIG. 5C).

Experiments described herein show that withacnistin is an inhibitor of the activation of STAT3 but not JAK2. In comparison, cucurbitacin A (Cuc A) was found to be an inhibitor of JAK2 but not STAT3 activation. Furthermore, withacnistin induces apoptosis and inhibits human tumor growth in mice. Finally, withacnistin induces apoptosis selectively in tumors that contain constitutively activated STAT3 but not in those tumors without activated STAT3.

In one aspect, the subject invention concerns a pharmaceutical composition comprising a withacnistin compound and a pharmaceutically acceptable carrier, adjuvant, diluent and/or excipient. In one embodiment, the composition comprises the compounds withacnistin, 3-methoxy-2,3-dihydrowithacnistin, or 3-ethoxy-2,3-dihydrowithacnistin, or a combination of two or all three of these compounds. Withacnistin is a potent suppressor of the JAK/STAT3 tumor survival pathway, and exhibits potent antitumor activity. In another aspect, the subject invention concerns a pharmaceutical composition comprising derivatives of withacnistin, 3-methoxy-2,3-dihydrowithacnistin, or 3-ethoxy-2,3-dihydrowithacnistin, such as those produced by treatment, extraction, or purification of these compounds with solvents such as ethanol or methanol. The pharmaceutical compositions of the subject invention are useful for treating cancer and inhibiting tumor growth, wherein the cancer or tumor is characterized by constitutive activation of the JAK2 and/or STAT3 signaling pathways.

The subject invention also concerns articles of manufacture useful in treating cancer and inhibiting tumor growth, wherein the cancer or tumor is characterized by constitutive activation of the JAK2 and/or STAT3 signaling pathways.

In another aspect, the subject invention concerns a method of inhibiting the growth of cancer cells in a patient by the administration of an effective amount of withacnistin compound locally (at the site of the cancer cells), or systemically. Preferably, a pharmaceutical composition of the invention is administered. Optionally, the method further comprises identifying a patient as one suffering from a cancer (e.g., tumor) that is characterized by constitutive activation of STAT3. For example, a biological sample (e.g., cells, cell extracts, serum, etc.) can be obtained from the patient (from a clinically relevant anatomical site) and analyzed for STAT3 activation prior to treatment with the withacnistin compound.

In a further aspect, the present invention concerns methods for modulating STAT3 activity in vitro or in vivo by administering an effective amount of a withacnistin compound. Preferably, a pharmaceutical composition of the invention is administered.

### BRIEF DESCRIPTION OF THE DRAWINGS

FIGS. 1A and 1B show structure-activity relationship (SAR) studies of cucurbitacins and the withacnistin mixture. Effects on signal transduction pathways in A549 cells are shown in FIG. 1A. A549 cells were treated with either vehicle control or cucurbitacin (Cuc) A, B, E, or I, or withacnistin mixture at 10 μM for 4 hours and cell lysates processed for immunoblotting with phospho-specific antibodies for STAT3, JAK2, Src, Erk1 (extracellular signal-regulated kinase), Erk2, JNK (c-Jun N-terminal kinase), and Akt (protein kinase B) antibodies as described under Materials and methods. FIG. 1A also indicates data obtained from both trypan blue exclusion assay and TUNEL staining (reported as average±s.d.), as described under Materials and Methods. Data are representative of at least three independent experiments. Referring to FIG. 1B, A549 cells were treated with either vehicle or withacnistin mixture for 4 hours and the lysates immunoprecipitated with anti-STAT3 antibody then immunoblotted with P-STAT3 and STAT3 antibodies as described under Materials and Methods. Data are representative of two independent experiments.

FIGS. 2A and 2B show that the withacnistin mixture induces apoptosis in human tumor cell lines and oncogene-transformed NIH 3T3 cells expressing constitutively activated STAT3. A549, MDA-MB-435, and MDA-MB-453 cells (shown in FIG. 2A) and Vector NIH 3T3, v-Src/3T3, and H-Ras/3T3 cells (shown in FIG. 2B) were treated with either vehicle control or 10 μM withacnistin mixture and processed for TUNEL staining as described under Materials and methods. Cells were costained with DAPI (4′,6-diamidino-2-phenylindole) to detect the nuclei. The table indicates induction of apoptosis by the withanistin mixture as determined by TUNEL assay.

FIG. 3 shows that the withacnistin mixture inhibits tumor growth in nude mice of both A549 human tumors cells and v-Src-transformed NIH 3T3 cells. Human lung adenocarcinoma A549 and v-Src-transformed NIH 3T3 cells were implanted s.c. onto the flanks of athymic nude mice. When the tumors reached an average size of 100-150 mm³, the animals were randomized and treated with either vehicle control (•) or 1 mg/kg/day of Cuc A (Δ), E (▴), I (∘), and withacnistin mixture (□) or 0.5 mg/kg/day Cuc B (⋄) as described under Materials and methods. **designates P<0.001 and *designates P<0.05.

FIGS. 4A-4D show immunohistochemical analysis of tumors for phosphotyrosine STAT3 and TUNEL staining. A549 tumor sections were stained as described under Materials and methods with P-STAT3 antibody and dTd (TUNEL) enzyme for the determination of cucurbitacin activity in the target tumor in vivo. Treatment conditions were: control (C); 1 mg/kg/day withacnistin mixture; 1 mg/kg/day Cuc I; 1 mg/kg/day Cuc A. Cells stained positive for phospho-STAT3 (shown in FIG. 4A) were scored and percent inhibition of STAT3 activation determined by comparison to vehicle control (shown in FIG. 4B). Cells stained positive for TUNEL (shown in FIG. 4C) were scored and induction of apoptosis determined by comparison to vehicle control (shown in FIG. 4D). For both graphs, *indicates P<0.05; **indicates P<0.005. Data were determined by counting sections from eight independent tumors. Data are representative of two independent experiments.

FIGS. 5A-5C show NMR and mass spectroscopy data demonstrating the chemical identity of NSC-135075. FIG. 5A shows the chemical structure of withacnistin, 3-methoxy-2,3-dihydrowithacnistin, and 3-ethoxy-2,3-dihydrowithacnistin, the mixture of which was identified from the NCI diversity set using a phosphotyrosine STAT3 high throughput cytoblot assay. FIG. 5B shows an NMR spectrum of NSC-135075, the main peak showing that the sample is withacnistin. The structure of withacnistin is also shown. FIG. 5C shows the mass spectrum of the main pure peak of NSC-135075, showing the expected peak corresponding to M+H at m/z 513.

FIG. 6 shows an NMR spectrum of NSC-135075, showing peaks consistent with a withacnistin structure, instead of cucurbitacin Q. The structure of withacnistin is also shown.

FIGS. 7A and 7B show that both the withacnistin mixture (mix) (a.k.a. NSC-135075), which was misidentified as cucurbitacin Q (CucQ), and pure withacnistin, inhibit P-STAT3 but not P-JAK2. Furthermore, pure withacnistin is more potent than the withacnistin mixture. FIG. 7A shows results from A549 cells following 4-hour treatment with withacnistin mix, pure withacnistin, withaferin A, or JSI-124. FIG. 7B shows results from MDA-MB468 cells following 4-hour treatment with withacnistin mix, pure withacnistin, withaferin A, or JSI-124.

FIGS. 8A-8C show that withacnistin suppresses P-STAT3 but not P-JAK2 levels, and is the active component of the NSC-135075 mixture (wit mix; wm).

FIGS. 9A-9D show that withacnistin inhibits IL-6, IFN-β, EGF, and PDGF stimulation of STAT3 but not STAT1 tyrosine phosphorylation in human cancer cell lines.

FIGS. 10A-10C show that withacnistin inhibits GM-CSF and PDGF stimulation of STAT5 tyrosine phosphorylation.

FIGS. 11A-11C show that withacnistin induces the levels of the STAT3 negative regulator SOCS3.

### DETAILED DISCLOSURE OF THE INVENTION

The subject invention pertains to compounds capable of interfering with the signaling events leading to the abnormally elevated levels of tyrosine phosphorylated STAT3 in many human cancers.

Constitutive activation of the JAK/STAT3 pathway is a major contributor to oncogenesis. Structure-activity relationship (SAR) studies with four cucurbitacin (Cuc) analogs, A, B, E, and I, led to the discovery that withacnistin inhibits the activation of STAT3 but not JAK2. Withacnistin inhibits selectively the activation of STAT3 and induces apoptosis without inhibition of JAK2, Src, Akt, Erk, or JNK activation. Furthermore, withacnistin induces apoptosis more potently in human and murine tumors that contain constitutively activated STAT3 (i.e., A549, MDA-MB-435, and v-Src/NIH 3T3) as compared to those that do not (i.e., H-Ras/NIH 3T3, MDA-MB-453, and NIH 3T3 cells). Finally, in a nude mouse tumor xenograft model, withacnistin suppresses tumor growth indicating that JAK2 inhibition is not sufficient to inhibit tumor growth and suggesting that the ability of withacnistin to inhibit tumor growth is related to its anti-STAT3 activity. These studies further validate STAT3 as a drug discovery target and provide evidence that pharmacological agents that can selectively reduce the P-STAT3 levels in human cancer cells result in tumor apoptosis and growth inhibition.

In one aspect, the subject invention concerns a pharmaceutical composition comprising the compounds withacnistin (Cherkaoui S. et al., Electrophoresis, 2003, 23(3): 336-342; Kaufmann B. et al., Phytochem. Anal., 2001, 12(5): 327-331; Kupchan S. M., J. Org. Chem., 1969, 34(12): 3858-3866), 3-methoxy-2,3-dihydrowithacnistin, or 3-ethoxy-2,3-dihydrowithacnistin, or a combination of two or all three of these compounds. The mixture of the three compounds is a potent suppressor of the STAT3 tumor survival pathway, and exhibits potent antitumor activity. In another aspect, the subject invention concerns a pharmaceutical composition comprising derivatives of withacnistin, 3-methoxy-2,3-dihydrowithacnistin, or 3-ethoxy-2,3-dihydrowithacnistin, such as those produced by treatment, extraction, or purification of these compounds with solvents such as ethanol or methanol. The pharmaceutical compositions of the subject invention are useful for treating cancer and inhibiting tumor growth, wherein the cancer or tumor is characterized by constitutive activation of the STAT3 signaling pathway.

As used herein, the terms “withacnistin compound” and “composition of the subject invention” refer to withacnistin, or a derivative thereof, or compositions containing them. In one embodiment, the composition comprises a mixture of withacnistin, 3-methoxy-2,3-dihydrowithacnistin, and 3-ethoxy-2,3-dihydrowithacnistin.

In one embodiment, the composition of the invention does not comprise 3-methoxy-2,3-dihydrowithacnistin.

In one embodiment, the composition of the invention does not comprise 3-ethoxy-2,3-dihydrowithacnistin.

In one embodiment, the composition of the invention does not comprise 3-methoxy-2,3-dihydrowithacnistin or 3-ethoxy-2,3-dihydrowithacnistin.

In one embodiment, the composition of the invention does not comprise a mixture of withacnistin, 3-methoxy-2,3-dihydrowithacnistin, and 3-ethoxy-2,3-dihydrowithacnistin.

In one embodiment, the composition of the invention does not consist of a mixture of withacnistin, 3-methoxy-2,3-dihydrowithacnistin, and 3-ethoxy-2,3-dihydrowithacnistin.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures therof, which preferably possesses the useful properties described herein, it being well known in the art how to prepare optically active forms and how to determine activity using the standard tests described herein, or using other similar tests which are well known in the art.

In another aspect, the subject invention concerns a method of inhibiting the growth of cancer cells in a patient by the administration of an effective amount of a withacnistin compound or a pharmaceutical composition comprising a withacnistin compound. Preferably, an effective amount of a pure or isolated withacnistin compound is administered. More preferably, an effective amount of pure or isolated withacnistin is administered. The method of the subject invention is useful in treating cancer and inhibiting tumor growth, wherein the cancer or tumor is characterized by constitutive activation of the STAT3 signaling pathway. Treatment of cancer involves a decrease of one or more symptoms associated with the particular cancer. Preferably, the treatment involves a decrease in tumor growth rate, particularly where the tumor is characterized by constitutive activation of the STAT3 signaling pathway.

According to the method of the subject invention, a withacnistin compound, or a pharmaceutically acceptable salt or analog thereof, is administered to a patient in an effective amount to decrease the constitutive levels of STAT3 activity. The withacinistin compound, or a pharmaceutically acceptable salt or analog thereof, can be administered prophylactically before tumor onset, or as treatment for existing tumors.

A withacnistin compound having the capability to modulate the STAT3 signaling pathway would be considered to have the desired biological activity in accordance with the subject invention. For therapeutic applications, an derivative of the subject invention preferably has the capability to inhibit activation STAT3 signaling pathway. Inhibition of STAT3 signaling by a withacnistin compound selectively promotes apoptosis in tumor cells that harbor constitutively activated STAT3. Therefore, the desirable goals of promoting apoptosis (“programmed cell death”) of selective cancerous cells and suppression of malignant transformation of normal cells within a patient are likewise accomplished through administration of antagonists or inhibitors of STAT 3 signaling of the present invention, which can be administered as simple compounds or in a pharmaceutical formulation.

The precise dosage will depend on a number of clinical factors, for example, the type of patient (such as human, non-human mammal, or other animal), age of the patient, and the particular cancer under treatment and its aggressiveness. A person having ordinary skill in the art would readily be able to determine, without undue experimentation, the appropriate dosages required to achieve the appropriate clinical effect.

A “patient” refers to a human, non-human mammal, or other animal in which inhibition of the STAT 3 signaling pathway would have a beneficial effect. Patients in need of treatment involving inhibition of the STAT 3 signaling pathway can be identified using standard techniques known to those in the medical profession.

As used herein, the term “treatment” includes amelioration or alleviation of a pathological condition and/or one or more symptoms thereof or curing such a condition.

The withacnistin compounds of the subject invention, including withacnistin, 3-methoxy-2,3-dihydrowithacnistin, and 3-ethoxy-2,3-dihydrowithacnistin, and derivatives of the foregoing, can be obtained through a variety of methods known in the art. For example, withacnistin can be isolated and purified from various sources. Derivatives of the subject invention can be synthesized using methods of organic synthesis known to those of ordinary skill in the art.

A further aspect of the present invention provides a method of modulating the activity of the STAT 3 signaling pathway and includes the step of contacting cells or tissue with an effective amount of a withacnistin compound, inhibiting activity of the STAT 3 signaling pathway. The method can be carried out in vivo or in vitro.

While the withacnistin compound can be administered as an isolated compound, it is preferred to administer these compounds as a pharmaceutical composition. The subject invention thus further provides pharmaceutical compositions comprising a withacnistin compound, as an active agent, or physiologically acceptable salt(s) thereof, in association with at least one pharmaceutically acceptable carrier or diluent. The pharmaceutical composition can be adapted for various routes of administration, such as enteral, parenteral, intravenous, intramuscular, topical, subcutaneous, and so forth. The withacnistin compound can be administered locally, at the site of the cancerous cells (e.g., intratumorally), or systemically. Administration can be continuous or at distinct intervals, as can be determined by a person of ordinary skill in the art.

The compounds of the subject invention can be formulated according to known methods for preparing pharmaceutically useful compositions. Formulations are described in a number of sources which are well known and readily available to those skilled in the art. For example, Remington's Pharmaceutical Science (Martin E. W., Easton Pennsylvania, Mack Publishing Company, 19^{th} ed., 1995) describes formulations which can be used in connection with the subject invention. Formulations suitable for administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The withacnistin compound of the present invention includes all hydrates and salts that can be prepared by those of skill in the art. Under conditions where the compounds of the present invention are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may incorporated into sustained-release preparations and devices.

According to the method of the subject invention, a withacnistin compound can be administered locally, at the site of cancer cells. For example, the withacnistin compound or composition can be directly administered to a tumor (e.g., topically or injected into the tumor).

According to the method of the subject invention, a withacnistin compound or a pharmaceutically acceptable salt or derivative thereof can be administered to a patient by itself, or co-administered with one or more other compounds, including one or more other withacnistin compounds, or a pharmaceutically acceptable salt or analog thereof. Co-administration can be carried out simultaneously (in the same or separate formulations) or consecutively. Furthermore, according to the method of the subject invention, the withacnistin compound, or a pharmaceutically acceptable salt or analog thereof, can be administered to a patient as adjunctive therapy. For example, a withacnistin compound, or a pharmaceutically acceptable salt or analog thereof, can be administered to a patient in conjunction with chemotherapy.

Thus, the withacnistin compounds of the subject invention, whether administered separately, or as a pharmaceutical composition, can include various other components as additives. Examples of acceptable components or adjuncts which can be employed in relevant circumstances include chemotherapeutic agents, anti-proliferative agents, anti-mitotic agents, anti-metabolite drugs, alkylating agents, drugs with target topoisomerases, drugs which target signal transduction in tumor cells, gene therapy, antisense agents, interfering RNA (RNAi), antibody therapeutics, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids, steroid analogues, and corticosteroids. Examples of chemotherapeutic agents are listed in Table 4. Such components can provide additional therapeutic benefit, act to affect the therapeutic action of the withacnistin compound, or act towards preventing any potential side effects which may be posed as a result of administration of the withacnistin compound. The withacnistin compounds of the subject invention can be conjugated to a therapeutic agent, as well.

TABLE 4
Examples of Chemotherapeutic Agents

| | |
|---|---|
| 13-cis-Retinoic Acid | Neosar |
| 2-Amino-6- | Neulasta |
| Mercaptopurine | Neumega |
| 2-CdA | Neupogen |
| 2-Chlorodeoxyadenosine | Nilandron |
| 5-fluorouracil | Nilutamide |
| 5-FU | Nitrogen Mustard |
| 6-TG | Novaldex |
| 6-Thioguanine | Novantrone |
| 6-Mercaptopurine | Octreotide |
| 6-MP | Octreotide acetate |
| Accutane | Oncospar |
| Actinomycin-D | Oncovin |
| Adriamycin | Ontak |
| Adrucil | Onxal |
| Agrylin | Oprevelkin |
| Ala-Cort | Orapred |
| Aldesleukin | Orasone |
| Alemtuzumab | Oxaliplatin |
| Alitretinoin | Paclitaxel |
| Alkaban-AQ | Pamidronate |
| Alkeran | Panretin |
| All-transretinoic acid | Paraplatin |
| Alpha interferon | Pediapred |
| Altretamine | PEG Interferon |
| Amethopterin | Pegaspargase |
| Amifostine | Pegfilgrastim |
| Aminoglutethimide | PEG-INTRON |
| Anagrelide | PEG-L-asparaginase |
| Anandron | Phenylalanine Mustard |
| Anastrozole | Platinol |
| Arabinosylcytosine | Platinol-AQ |
| Ara-C | Prednisolone |
| Aranesp | Prednisone |
| Aredia | Prelone |
| Arimidex | Procarbazine |
| Aromasin | PROCRIT |
| Arsenic trioxide | Proleukin |
| Asparaginase | Prolifeprospan 20 with Carmustine implant |
| ATRA | Purinethol |
| Avastin | Raloxifene |
| BCG | Rheumatrex |
| BCNU | Rituxan |
| Bevacizumab | Rituximab |
| Bexarotene | Roveron-A (interferon alfa-2a) |
| Bicalutamide | Rubex |
| BiCNU | Rubidomycin hydrochloride |
| Blenoxane | Sandostatin |
| Bleomycin | Sandostatin LAR |
| Bortezomib | Sargramostim |
| Busulfan | Solu-Cortef |
| Busulfex | Solu-Medrol |
| C225 | STI-571 |
| Calcium Leucovorin | Streptozocin |
| Campath | Tamoxifen |
| Camptosar | Targretin |
| Camptothecin-11 | Taxol |
| Capecitabine | Taxotere |
| Carac | Temodar |
| Carboplatin | Temozolomide |
| Carmustine | Teniposide |
| Carmustine wafer | TESPA |
| Casodex | Thalidomide |
| CCNU | Thalomid |
| CDDP | TheraCys |
| CeeNU | Thioguanine |
| Cerubidine | Thioguanine Tabloid |
| cetuximab | Thiophosphoamide |
| Chlorambucil | Thioplex |
| Cisplatin | Thiotepa |
| Citrovorum Factor | TICE |
| Cladribine | Toposar |
| Cortisone | Topotecan |
| Cosmegen | Toremifene |
| CPT-11 | Trastuzumab |
| Cyclophosphamide | Tretinoin |
| Cytadren | Trexall |
| Cytarabine | Trisenox |
| Cytarabine liposomal | TSPA |
| Cytosar-U | VCR |
| Cytoxan | Velban |
| Dacarbazine | Velcade |
| Dactinomycin | VePesid |
| Darbepoetin alfa | Vesanoid |
| Daunomycin | Viadur |
| Daunorubicin | Vinblastine |
| Daunorubicin | Vinblastine Sulfate |
| hydrochloride | Vincasar Pfs |
| Daunorubicin liposomal | Vincristine |
| DaunoXome | Vinorelbine |
| Decadron | Vinorelbine tartrate |
| Delta-Cortef | VLB |
| Deltasone | VP-16 |
| Denileukin diftitox | Vumon |
| DepoCyt | Xeloda |
| Dexamethasone | Zanosar |
| Dexamethasone acetate | Zevalin |
| dexamethasone sodium | Zinecard |
| phosphate | Zoladex |
| Dexasone | Zoledronic acid |
| Dexrazoxane | Zometa |
| DHAD | Gliadel wafer |
| DIC | Glivec |
| Diodex | GM-CSF |
| Docetaxel | Goserelin |
| Doxil | granulocyte - colony stimulating factor |
| Doxorubicin | Granulocyte macrophage colony stimulating |
| Doxorubicin liposomal | factor |
| Droxia | Halotestin |
| DTIC | Herceptin |
| DTIC-Dome | Hexadrol |
| Duralone | Hexalen |
| Efudex | Hexamethylmelamine |
| Eligard | HMM |
| Ellence | Hycamtin |
| Eloxatin | Hydrea |
| Elspar | Hydrocort Acetate |
| Emcyt | Hydrocortisone |
| Epirubicin | Hydrocortisone sodium phosphate |
| Epoetin alfa | Hydrocortisone sodium succinate |
| Erbitux | Hydrocortone phosphate |
| Erwinia L-asparaginase | Hydroxyurea |
| Estramustine | Ibritumomab |
| Ethyol | Ibritumomab Tiuxetan |
| Etopophos | Idamycin |
| Etoposide | Idarubicin |
| Etoposide phosphate | Ifex |
| Eulexin | IFN-alpha |
| Evista | Ifosfamide |
| Exemestane | IL-2 |
| Fareston | IL-11 |
| Faslodex | Imatinib mesylate |
| Femara | Imidazole Carboxamide |
| Filgrastim | Interferon alfa |
| Floxuridine | Interferon Alfa-2b (PEG conjugate) |
| Fludara | Interleukin - 2 |
| Fludarabine | Interleukin-11 |
| Fluoroplex | Intron A (interferon alfa-2b) |
| Fluorouracil | Leucovorin |
| Fluorouracil (cream) | Leukeran |
| Fluoxymesterone | Leukine |
| Flutamide | Leuprolide |
| Folinic Acid | Leurocristine |
| FUDR | Leustatin |
| Fulvestrant | Liposomal Ara-C |
| G-CSF | Liquid Pred |
| Gefitinib | Lomustine |
| Gemcitabine | L-PAM |
| Gemtuzumab ozogamicin | L-Sarcolysin |
| Gemzar | Meticorten |
| Gleevec | Mitomycin |
| Lupron | Mitomycin-C |
| Lupron Depot | Mitoxantrone |
| Matulane | M-Prednisol |
| Maxidex | MTC |
| Mechlorethamine | MTX |
| Mechlorethamine | Mustargen |
| Hydrochlorine | Mustine |
| Medralone | Mutamycin |
| Medrol | Myleran |
| Megace | Iressa |
| Megestrol | Irinotecan |
| Megestrol Acetate | Isotretinoin |
| Melphalan | Kidrolase |
| Mercaptopurine | Lanacort |
| Mesna | L-asparaginase |
| Mesnex | LCR |
| Methotrexate | |
| Methotrexate Sodium | |
| Methylprednisolone | |
| Mylocel | |
| Letrozole | |

Additional agents that can co-administered to a patient in the same or as a separate formulation include those that modify a given biological response, such as immunomodulators. For example, proteins such as tumor necrosis factor (TNF), interferon (such as alpha-interferon and beta-interferon), nerve growth factor (NGF), platelet derived growth factor (PDGF), and tissue plasminogen activator can be administered. Biological response modifiers, such as lymphokines, interleukins (such as interleukin-1 (IL-1), interleukin-2 (IL-2), and interleukin-6 (IL-6)), granulocyte macrophage colony stimulating factor (GM-CSF), granulocyte colony stimulating factor (G-CSF), or other growth factors can be administered.

The subject invention also provides an article of manufacture useful in treating cancer characterized by constitutive activation of the STAT 3 signaling pathway. The article contains a pharmaceutical composition containing a withacnistin compound, and a pharmaceutically acceptable carrier or diluent. The article of manufacture can be, for example, a vial, bottle, intravenous bag, syringe, nasal applicator, microdialysis probe, or other container for the pharmaceutical composition. The nasal applicator containing the pharmaceutical composition of the invention can further comprise a propellent. The article of manufacture can further comprise packaging. The article of manufacture can also include printed material disclosing instructions for concerning administration of the pharmaceutical composition for the treatment of cancer. Preferably, the printed material discloses instructions concerning administration of the pharmaceutical composition for the treatment of cancer characterized by constitutive activation of the STAT 3 signaling pathway. The printed material can be embossed or imprinted on the article of manufacture and indicate the amount or concentration of the active agent (withacnistin compound), recommended doses for treatment of the cancer, or recommended weights of individuals to be treated.

As used herein, the terms “pure” or “isolated” refer to a composition that includes at least 85% or 90% by weight, preferably 95% to 98% by weight, and even more preferably 99% to 100% by weight, of the withacnistin compound, the remainder comprising other chemical species or enantiomers.

As used herein, the terms “cancer” and “cancerous” refer to or describe the physiological condition in mammals that is typically characterized by unregulated cell growth, i.e., proliferative disorders. Examples of such proliferative disorders include cancers such as carcinoma, lymphoma, blastoma, sarcoma, and leukemia, as well as other cancers disclosed herein. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); pancreatic cancer; retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas. Examples of cancer types are listed in Table 3.

TABLE 3
Examples of Cancer Types

| | |
|---|---|
| Acute Lymphoblastic Leukemia, | Hairy Cell Leukemia |
| Adult | Head and Neck Cancer |
| Acute Lymphoblastic Leukemia, | Hepatocellular (Liver) Cancer, |
| Childhood | Adult (Primary) |
| Acute Myeloid Leukemia, Adult | Hepatocellular (Liver) Cancer, |
| Acute Myeloid Leukemia, | Childhood (Primary) |
| Childhood | Hodgkin's Lymphoma, Adult |
| Adrenocortical Carcinoma | Hodgkin's Lymphoma, Childhood |
| Adrenocortical Carcinoma, | Hodgkin's Lymphoma During |
| Childhood | Pregnancy |
| AIDS-Related Cancers | Hypopharyngeal Cancer |
| AIDS-Related Lymphoma | Hypothalamic and Visual Pathway |
| Anal Cancer | Glioma, Childhood |
| Astrocytoma, Childhood Cerebellar | Intraocular Melanoma |
| Astrocytoma, Childhood Cerebral | Islet Cell Carcinoma (Endocrine |
| Basal Cell Carcinoma | Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, | Laryngeal Cancer |
| Osteosarcoma/Malignant Fibrous | Laryngeal Cancer, Childhood |
| Histiocytoma | Leukemia, Acute Lymphoblastic, |
| Brain Stem Glioma, Childhood | Adult |
| Brain Tumor, Adult | Leukemia, Acute Lymphoblastic, |
| Brain Tumor, Brain Stem Glioma, | Childhood |
| Childhood | Leukemia, Acute Myeloid, Adult |
| Brain Tumor, Cerebellar | Leukemia, Acute Myeloid, |
| Astrocytoma, Childhood | Childhood |
| Brain Tumor, Cerebral | Leukemia, Chronic Lymphocytic |
| Astrocytoma/Malignant Glioma, | Leukemia, Chronic Myelogenous |
| Childhood | Leukemia, Hairy Cell |
| Brain Tumor, Ependymoma, | Lip and Oral Cavity Cancer |
| Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial | Lung Cancer, Small Cell |
| Primitive Neuroectodermal | Lymphoma, AIDS-Related |
| Tumors, Childhood | Lymphoma, Burkitt's |
| Brain Tumor, Visual Pathway and | Lymphoma, Cutaneous T-Cell, see |
| Hypothalamic Glioma, Childhood | Mycosis Fungoides and Sézary |
| Brain Tumor, Childhood | Syndrome |
| Breast Cancer | Lymphoma, Hodgkin's, Adult |
| Breast Cancer, Childhood | Lymphoma, Hodgkin's, |
| Breast Cancer, Male | Childhood |
| Bronchial Adenomas/Carcinoids, | Lymphoma, Hodgkin's During |
| Childhood | Pregnancy |
| Burkitt's Lymphoma | Lymphoma, Non-Hodgkin's, Adult |
| Carcinoid Tumor, Childhood | Lymphoma, Non-Hodgkin's, |
| Carcinoid Tumor, Gastrointestinal | Childhood |
| Carcinoma of Unknown Primary | Lymphoma, Non-Hodgkin's |
| Central Nervous System | During Pregnancy |
| Lymphoma, Primary | Lymphoma, Primary Central |
| Cerebellar Astrocytoma, Childhood | Nervous System |
| Cerebral Astrocytoma/Malignant | Macroglobulinemia, Waldenström's |
| Glioma, Childhood | Malignant Fibrous Histiocytoma of |
| Cervical Cancer | Bone/Osteosarcoma |
| Childhood Cancers | Medulloblastoma, Childhood |
| Chronic Lymphocytic Leukemia | Melanoma |
| Chronic Myelogenous Leukemia | Melanoma, Intraocular (Eye) |
| Chronic Myeloproliferative | Merkel Cell Carcinoma |
| Disorders | Mesothelioma, Adult Malignant |
| Colon Cancer | Mesothelioma, Childhood |
| Colorectal Cancer, Childhood | Metastatic Squamous Neck Cancer |
| Cutaneous T-Cell Lymphoma, see | with Occult Primary |
| Mycosis Fungoides and Sézary | Multiple Endocrine Neoplasia |
| Syndrome | Syndrome, Childhood |
| Endometrial Cancer | Multiple Myeloma/Plasma Cell |
| Ependymoma, Childhood | Neoplasm |
| Esophageal Cancer | Mycosis Fungoides |
| Esophageal Cancer, Childhood | Myelodysplastic Syndromes |
| Ewing's Family of Tumors | Myelodysplastic/Myeloproliferative |
| Extracranial Germ Cell Tumor, | Diseases |
| Childhood | Myelogenous Leukemia, Chronic |
| Extragonadal Germ Cell Tumor | Myeloid Leukemia, Adult Acute |
| Extrahepatic Bile Duct Cancer | Myeloid Leukemia, Childhood Acute |
| Eye Cancer, Intraocular Melanoma | Myeloma, Multiple |
| Eye Cancer, Retinoblastoma | Myeloproliferative Disorders, |
| Gallbladder Cancer | Chronic |
| Gastric (Stomach) Cancer | Nasal Cavity and Paranasal Sinus |
| Gastric (Stomach) Cancer, | Cancer |
| Childhood | Nasopharyngeal Cancer |
| Gastrointestinal Carcinoid Tumor | Nasopharyngeal Cancer, Childhood |
| Germ Cell Tumor, Extracranial, | Neuroblastoma |
| Childhood | Non-Hodgkin's Lymphoma, Adult |
| Germ Cell Tumor, Extragonadal | Non-Hodgkin's Lymphoma, |
| Germ Cell Tumor, Ovarian | Childhood |
| Gestational Trophoblastic Tumor | Non-Hodgkin's Lymphoma During |
| Glioma, Adult | Pregnancy |
| Glioma, Childhood Brain Stem | Non-Small Cell Lung Cancer |
| Glioma, Childhood Cerebral | Oral Cancer, Childhood |
| Astrocytoma | Oral Cavity Cancer, Lip and |
| Glioma, Childhood Visual Pathway | Oropharyngeal Cancer |
| and Hypothalamic | Osteosarcoma/Malignant Fibrous |
| Skin Cancer (Melanoma) | Histiocytoma of Bone |
| Skin Carcinoma, Merkel Cell | Ovarian Cancer, Childhood |
| Small Cell Lung Cancer | Ovarian Epithelial Cancer |
| Small Intestine Cancer | Ovarian Germ Cell Tumor |
| Soft Tissue Sarcoma, Adult | Ovarian Low Malignant Potential |
| Soft Tissue Sarcoma, Childhood | Tumor |
| Squamous Cell Carcinoma, | Pancreatic Cancer |
| see Skin Cancer (non-Melanoma) | Pancreatic Cancer, Childhood |
| Squamous Neck Cancer with | Pancreatic Cancer, Islet Cell |
| Occult Primary, Metastatic | Paranasal Sinus and Nasal Cavity |
| Stomach (Gastric) Cancer | Cancer |
| Stomach (Gastric) Cancer, | Parathyroid Cancer |
| Childhood | Penile Cancer |
| Supratentorial Primitive | Pheochromocytoma |
| Neuroectodermal Tumors, | Pineoblastoma and Supratentorial |
| Childhood | Primitive Neuroectodermal |
| T-Cell Lymphoma, Cutaneous, see | Tumors, Childhood |
| Mycosis Fungoides and Sézary | Pituitary Tumor |
| Syndrome | Plasma Cell Neoplasm/Multiple |
| Testicular Cancer | Myeloma |
| Thymoma, Childhood | Pleuropulmonary Blastoma |
| Thymoma and Thymic Carcinoma | Pregnancy and Breast Cancer |
| Thyroid Cancer | Pregnancy and Hodgkin's Lymphoma |
| Thyroid Cancer, Childhood | Pregnancy and Non-Hodgkin's |
| Transitional Cell Cancer of the | Lymphoma |
| Renal Pelvis and Ureter | Primary Central Nervous System |
| Trophoblastic Tumor, Gestational | Lymphoma |
| Unknown Primary Site, Carcinoma | Prostate Cancer |
| of, Adult | Rectal Cancer |
| Unknown Primary Site, Cancer of, | Renal Cell (Kidney) Cancer |
| Childhood | Renal Cell (Kidney) Cancer, |
| Unusual Cancers of Childhood | Childhood |
| Ureter and Renal Pelvis, | Renal Pelvis and Ureter, Transitional |
| Transitional Cell Cancer | Cell Cancer |
| Urethral Cancer | Retinoblastoma |
| Uterine Cancer, Endometrial | Rhabdomyosarcoma, Childhood |
| Uterine Sarcoma | Salivary Gland Cancer |
| Vaginal Cancer | Salivary Gland Cancer, Childhood |
| Visual Pathway and Hypothalamic | Sarcoma, Ewing's Family of Tumors |
| Glioma, Childhood | Sarcoma, Kaposi's |
| Vulvar Cancer | Sarcoma, Soft Tissue, Adult |
| Waldenström's Macroglobulinemia | Sarcoma, Soft Tissue, Childhood |
| Wilms' Tumor | Sarcoma, Uterine |
| | Sezary Syndrome |
| | Skin Cancer (non-Melanoma) |
| | Skin Cancer, Childhood |

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

As used herein, the term “apoptosis”, or programmed cell death, refers to the process in which the cell undergoes a series of molecular events leading to some or all of the following morphological changes: DNA fragmentation; chromatin condensation; nuclear envelope breakdown; and cell shrinkage.

As used herein, the term “STAT” refers to signal transducers and activators of transcription, which represent a family of proteins that, when activated by protein tyrosine kinases in the cytoplasm of the cell, migrate to the nucleus and activate gene transcription. Examples of mammalian STATs include STAT 1, STAT2, STAT3, STAT4, STAT5a, STAT5b, and STAT6.

As used herein, the term “signaling” and “signaling transduction” represents the biochemical process involving transmission of extracellular stimuli, via cell surface receptors through a specific and sequential series of molecules, to genes in the nucleus resulting in specific cellular responses to the stimuli.

As used herein, the term “constitutive activation,” as in the constitutive activation of the STAT pathway, refers to a condition where there is an abnormally elevated level of tyrosine phosphorylated STAT3 within a given cancer cell(s), as compared to a corresponding normal (non-cancer or non-transformed) cell. Constitutive activation of STAT3 has been exhibited in a large variety of malignancies, including, for example, breast carcinoma cell lines; primary breast tumor specimens; ovarian cancer cell lines and tumors; multiple myeloma tumor specimens; blood malignancies, such as acute myelogenous leukemia; and breast carcinoma cells, as described in published PCT international application WO 00/44774 (Jove, R. et al.), the disclosure of which is incorporated herein by reference in its entirety. In one embodiment, the cancer to be treated is not the cancer type of the nasopharynx (KB) cell line (Kupchan, S. M. et al. J. Org. Chem., 1969, 34(12): 3858-3866, which is incorporated herein by reference in its entirety).

Methods for determining whether a human or non-human mammalian patient has abnormally high levels of constitutively-activated STAT3 are known in the art and are described, for example, in U.S. patent publication 2004-0138189-A1 and PCT publication 02/078617 A, each of which are incorporated herein by reference in their entirety. Optionally, the methods of the invention further comprise identifying a patient suffering from a condition (e.g., cancer) associated with an abnormally elevated level of tyrosine phosphorylated STAT3, or determining whether the cancer cells can be characterized as having abnormally elevated levels of tyrosine phosphorylated STAT3.

As used herein, the term “pharmaceutically acceptable salt or prodrug” is intended to describe any pharmaceutically acceptable form (such as an ester, phosphate ester, salt of an ester or a related group) of a withacnistin compound, which, upon administration to a patient, provides the withacnistin compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The following embodiments are included in this invention:

Embodiment 1: a method for treating cancer in a patient, the method comprising administering withacnistin, or a pharmaceutically acceptable salt or analog thereof, to a patent in need of treatment.

Embodiment 2: a method for treating cancer in a patient, the method comprising administering a pharmaceutical composition comprising a P-STAT inhibitor to the patient, the P-STAT inhibitor consisting essentially of withacnistin.

Embodiment 3: a method for inhibiting the growth of cancer cells in a patient, the method comprising administering a pharmaceutical composition comprising a P-STAT inhibitor to the patient, the P-STAT inhibitor consisting essentially of withacnistin, resulting in inhibited cancer growth.

Embodiment 4: a method for treating cancer in a patient, the method comprising administering a pharmaceutical composition comprising only one withacnistin compound, wherein the withacnistin compound is withacnistin or a pharmaceutically acceptable salt thereof.

Embodiment 5: the method of any of embodiments 1-4, further comprising identifying the patient as one suffering from cancer characterized by constitutive activation of the STAT3 signaling pathway.

Embodiment 6: the method of any of embodiments 1-4, wherein the cancer cells are characterized by constitutive activation of the STAT3 signaling pathway.

Embodiment 7: the method of any of embodiments 1-4, wherein the cancer is selected from the group consisting of lung cancer, colon cancer, pancreatic cancer, ovarian cancer, and breast cancer.

Embodiment 8: the method of any of embodiments 2-4, wherein the pharmaceutical composition inhibits the STAT3 signaling pathway, but does not inhibit the JAK2 signaling pathway.

Embodiment 9: the method of any of embodiments 2-4, wherein the cancer is characterized by abnormal STAT3 pathway activity.

Embodiment 10: the method of any of embodiments 1-4, wherein the patient is suffering from a tumor and the compound inhibits growth of the tumor.

Embodiment 11: the method of any of embodiments 1-4, wherein the route of the administration is selected from the group consisting of intravenous, intramuscular, oral, and intra-nasal.

Embodiment 12: a pharmaceutical composition comprising isolated withacnistin, and a pharmaceutically acceptable carrier or diluent.

Embodiment 13: the pharmaceutical composition of embodiment 12, wherein the composition further comprises an immunomodulating agent.

Embodiment 14: the pharmaceutical composition of embodiment 12, wherein the composition further comprises an agent selected from the group consisting of an antioxidant, free radical scavenging agent, peptide, growth factor, antibiotic, bacteriostatic agent, immunosuppressive, anticoagulant, buffering agent, anti-inflammatory agent, anti-pyretic, time-release binder, anesthetic, steroid, and corticosteroid.

Embodiment 15: a method for preparing a pharmaceutical composition, the method comprising isolating withacnistin from a plant and combining the isolated withacnistin with a pharmaceutically acceptable carrier or diluent.

Embodiment 16: a pharmaceutical composition containing a therapeutically effective amount of withacnistin or a physiologically acceptable salt or prodrug thereof, in admixture with one, or more, pharmaceutically acceptable carriers, adjuvants, diluents and/or excipients.

Embodiment 17: the pharmaceutical composition of embodiment 16, wherein the withacnistin is in crystalline form.

Embodiment 18: the pharmaceutical composition of embodiment 16, wherein the withacnistin is in the form of an amorphous solid.

Embodiment 19: the pharmaceutical composition of any of embodiments 16-18, further comprising a second active pharmaceutical ingredient (API).

Embodiment 20: the pharmaceutical composition of embodiment 19, wherein the second API is an anti-cancer compound.

Embodiment 21: a pharmaceutical composition comprising a co-crystal comprising withacnistin and a co-crystal former.

Embodiment 22: the pharmaceutical composition of embodiment 21, wherein the co-crystal further comprises a second active pharmaceutical ingredient (API).

Embodiment 23: the pharmaceutical composition of embodiment 22, wherein the second API is an anti-cancer compound.

Embodiment 24: a method of treating cancer in a patient, the method comprising administering to the patient a therapeutically effective amount of the pharmaceutical composition of one of embodiments 16-23.

Embodiment 25: the method of embodiment 24, wherein the cancer cells are characterized by constitutive activation of the STAT3 signaling pathway.

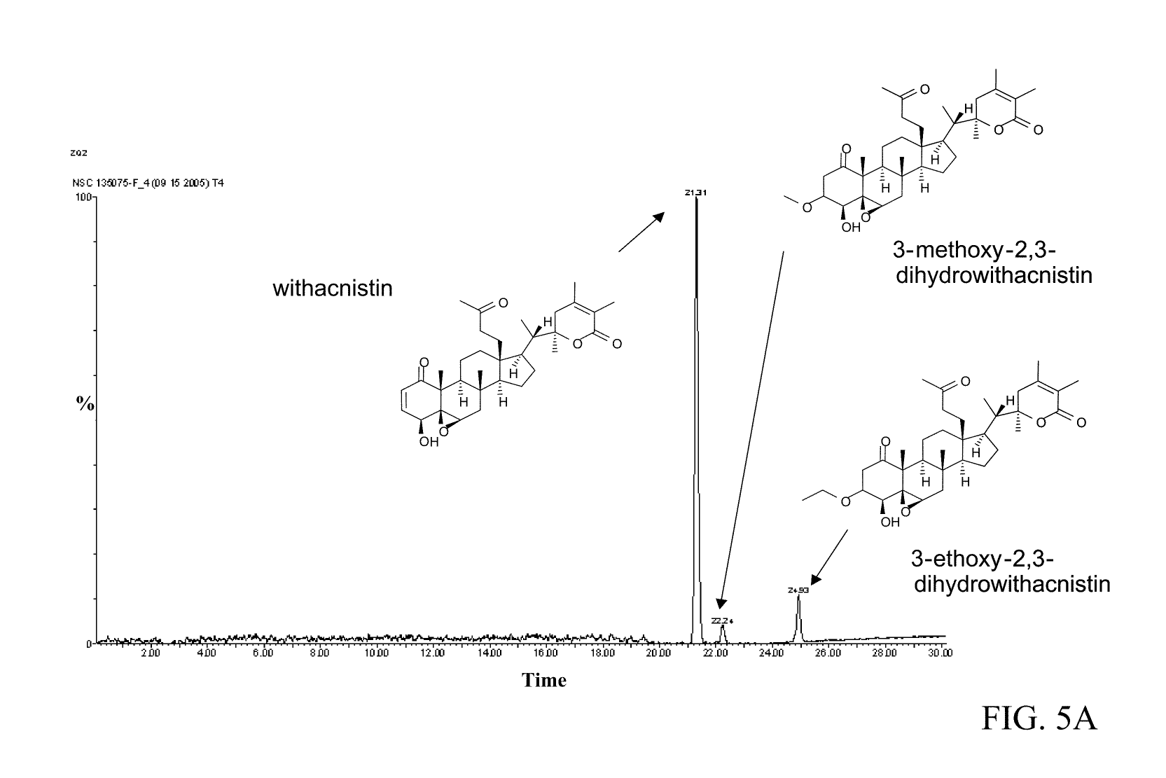

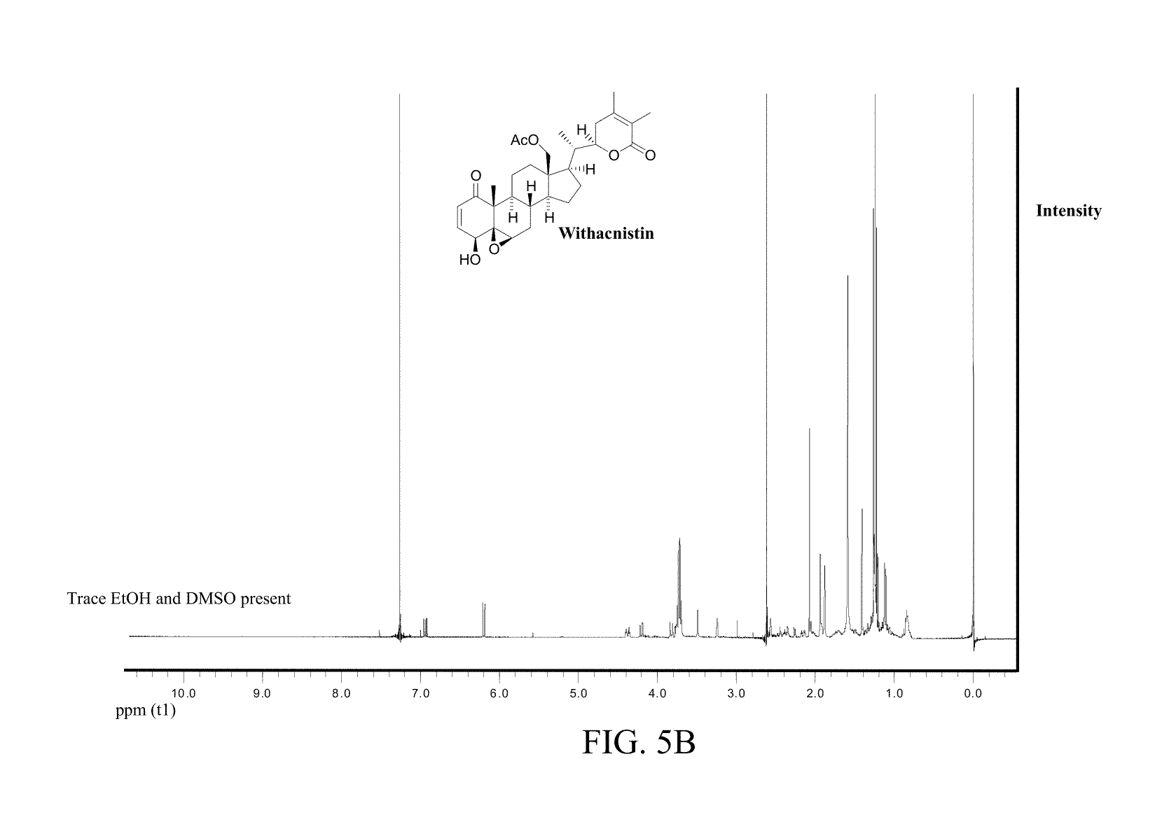

All experimental data disclosed in the publication Sun J. et al., (Sun J. et al., Oncogene, “Cucurbitacin Q: a selective STAT3 activation inhibitor with potent antitumor activity”, 2005 May, 24 (20): 3236-3245, which is incorporated herein by reference in its entirety) that references “cucurbitacin Q” actually pertains to a mixture of withacnistin, 3-methoxy-2,3-dihydrowithacnistin, and 3-ethoxy-2,3-dihydrowithacnistin, as shown in FIG. 5.

All patents, patent applications, provisional applications, and publications referred to or cited herein, supra or infra, are incorporated by reference in their entirety, including all figures and tables, to the extent they are not inconsistent with the explicit teachings of this specification.

Materials and Methods

Cell lines. All human tumor cell lines used were obtained from American Type Culture Collection (Manassas, Va., USA). Stably transfected v-Src/NIH 3T3 cell line has been described earlier (Turkson, J. et al. Mol. Cell. Biol., 1999, 19: 7519-7528).

Cucurbitacin analogs. All cucurbitacin compounds were obtained from the National Cancer Institute: cucurbitacin A (NSC #94743), cucurbitacin B (NSC #49451), cucurbitacin E (NSC #106399), cucurbitacin I (NSC #521777).

Withacnistin. The withacnistin mixture (NSC #135075) of FIG. 5 was obtained from the National Cancer Institute.

Western blotting. Treated cell samples were lysed in 30 mM HEPES, pH 7.5, 10 mM NaCl, 5 mM MgCl₂, 25 mM NaF, 1 mM EGTA, 1% Triton X-100, 10% glycerol, 2 mM sodium orthovanadate, 10 μg/ml aprotinin, 10 μ/ml soybean trypsin inhibitor, 25 μg/ml leupeptin, 2 mM PMSF, and 6.4 mg/ml p-nitrophenylphosphate. Phospho-STAT3, phospho-AKT, phospho-Src, and phospho-p42/p44 MAPK antibodies were obtained from Cell Signaling Technologies (Cambridge, Mass., USA). Phospho-JNK and whole STAT3 antibodies were purchased from Santa Cruz Biotechnology (Santa Cruz, Calif., USA); phospho-JAK2 antibody came from Upstate Biotechnology (Lake Placid, N.Y., USA). Membranes were blocked in either 5% milk in phosphate-buffered saline (PBS), pH 7.4, containing 0.1% Tween-20 (PBS-T) or 1% BSA in tris-buffered saline (TBS), pH 7.5, containing 0.1% Tween-20 (TBS-T). Phospho-specific antibodies (excepting P-MAPK and P-JNK) were incubated in 1% BSA in TBS-T while all other antibodies were diluted in 5% milk in PBS-T for either 2 h at room temperature or overnight at 4° C. HRP-conjugated secondary antibodies (Jackson ImmunoResearch, West Grove, Pa., USA) were diluted in 5% milk in either PBS-T or TBS-T at 1:1000 dilution for 1 h at room temperature. Western blots were visualized using enhanced chemiluminescence.

STAT3 immunoprecipitation. A549 cells were treated for 4 hour with vehicle or the withacnistin mixture, then lysed in 150 mM HEPES, pH 7.5, 150 mM NaCl, 1 mM EDTA, 0.5% NP-40, 10% glycerol, 5 mM NaF, 1 mM DTT, 1 mM PMSF, 2 mM sodium orthovanadate, and 5 μg/ml leupeptin. Sample lysates were collected and cleared, then 500 μg of lysate was immunoprecipitated with 50 ng STAT3 antibody overnight at 4° C., then rocked with 25 μl Protein A/G PLUS agarose (Santa Cruz Biotechnology) for 1 hour at 4° C. Samples were washed four times with lysis buffer, then boiled in 2×SDS-PAGE sample buffer and run on 10% SDS-PAGE gel. Protein was transferred to nitrocellulose then blotted as above for both phospho-specific STAT3 and STAT3.

Antitumor activity in the nude mouse tumor xenograft model. Nude mice (Charles River, Wilmington, Mass., USA) were maintained in accordance with the Institutional Animal Care and Use Committee (IACUC) procedures and guidelines. A549 cells were harvested, resuspended in PBS, and injected subcutaneously (s.c.) into the right and left flank (1×10⁷ cells per flank) of 8-week-old female nude mice as reported previously (Blaskovich, M. A. et al. Cancer Res., 2003, 63: 1270-1279). When tumors reached about 150 mm³, animals were randomized (four animals per group; two tumors per animal) and dosed intraperitoneally (i.p.) either with cucurbitacin analogs (0.5 or 1 mg/kg/day, i.p.) in 20% DMSO in water or with an equal volume of vehicle control. The tumor volumes were determined by measuring the length (l) and the width (w) and calculating the volume (V=lw²/2) as described previously (Blaskovich, M. A. et al. Cancer Res., 2003, 63: 1270-1279). Statistical significance between control and treated animals were evaluated by using Student's t-test.

In vitro cellular proliferation and TUNEL assays. Subconfluent A549, MDA-MB-435, MDA-MB-453, MDAMB-468, v-Src transformed NIH 3T3 (v-Src/3T3), H-Ras transformed NIH 3T3 (H-Ras/3T3), and vector NIH 3T3 cells were grown in the presence of 10 μM cucurbitacin A, cucurbitacin I, withacnistin mixture, or DMSO vehicle control. After 24 hours, cells were harvested by trypsinization and counted via trypan blue exclusion assay to determine cellular viability. In all, 75,000-150,000 cells (depending on cell line) were then spun onto glass slides using a Cytospin 3 centrifuge (Thermo Shandon Inc., Pittsburgh, Pa., USA). After fixing cells to the slides with 4% paraformaldehyde in PBS, pH 7.5, for 1 h at room temperature, cells were labeled for apoptotic DNA strand breaks by TUNEL reaction using an in situ cell death detection kit (Roche Applied Science, Indianapolis, Ind., USA) according to the manufacturer's instructions, then mounted in Vectashield mounting medium (Vector Laboratories, Burlingame, Calif., USA) containing 4′,6-diamidino-2-phenylindole (DAPI) to counterstain DNA. Fluorescein-labeled DNA strand breaks (TUNEL-positive cells) were then visualized using a fluorescent microscope (Leica Microsystems Inc., Bannockburn, Ill., USA) and pictures taken with a digital camera (Diagnostic Instruments, Inc., Sterling Heights, Mich., USA). TUNELpositive nuclei were counted and compared to DAPI-stained nuclei to determine the percent induction of apoptosis by the different cucurbitacin compounds. Statistical significance between control and treated tumors were evaluated by using Student's t-test.

P-STAT3 immunohistochemistry. On the termination day of the A549 antitumor experiment, tumors were extracted and fixed in 10% neutral-buffered formalin for 6 hours. After fixation, the tissue samples were processed into paraffin blocks. Tissue sections (5 μm) were dewaxed with xylene and rehydrated through descending alcohol to deionized water and then placed in PBS. Antigens were retrieved briefly with citrate buffer, pH 6.0, in a microwave followed by a mild trypsinization (0.025% trypsin in 50 mM Tris buffer containing 0.05% calcium chloride, pH 7.6). From this point, all steps were carried out in a DAKO Autostainer (DakoCytomation California, Inc., Carpinteria, Calif., USA). Sections were rinsed three times in TBS-Tween buffer, pH 7.6, then endogenous peroxidases were quenched with 3% hydrogen peroxide and nonspecific binding with 2% normal goat serum in 3% BSA/PBS. Sections then were incubated overnight with 1:400 phospho-STAT3 (Cell Signaling Technologies) at 4° C. in a humidified chamber. Detection was performed using the Elite ABC Rabbit kit (Vector Laboratories) and DAB chromogen (DakoCytomation California, Inc.) according to the manufacturer's instructions. Slides were counterstained for 20-30 seconds with modified Mayer's hematoxylin, dehydrated through ascending alcohol, cleared, and mounted with resinous mounting medium. Quantification was performed by counting both the phospho-STAT3-positive and -negative cells on slides representative of eight tumors and significance was determined by Student's t-test.

TUNEL immunohistochemistry. Tumors were harvested, frozen, and dewaxed as described for P-STAT3 immunohistochemistry. Tissue sections (5 μm) were digested for 10 minutes with 25 μg/ml proteinase K in PBS and then washed thoroughly. Peroxidases were quenched with 3% hydrogen peroxide in PBS and washed. Sections were equilibrated with equilibration buffer, then incubated in 30% TdT enzymes/70% digoxigenin nucleotidyl reaction buffer for 1 hour at 37° C. in a humidified chamber. The labeling reaction was stopped in stop/wash buffer with moderate shaking. Slides then were placed on the Dako Autostainer and incubated with antidigoxigenin-peroxidase (DakoCytomation California, Inc.) for 30 minutes using DAB substrate. Sections were counterstained with methyl green (Vector Laboratories), dehydrated through ascending alcohol, cleared, and mounted with resinous mounting medium. The quantification was performed by counting both the TUNEL-positive and -negative cells on slides representative of eight tumors and significance was determined by Student's t-test.

### EXAMPLE 1

### Withacnistin Selectively Suppresses STAT3 but not JAK2 Activation in A549 Cells

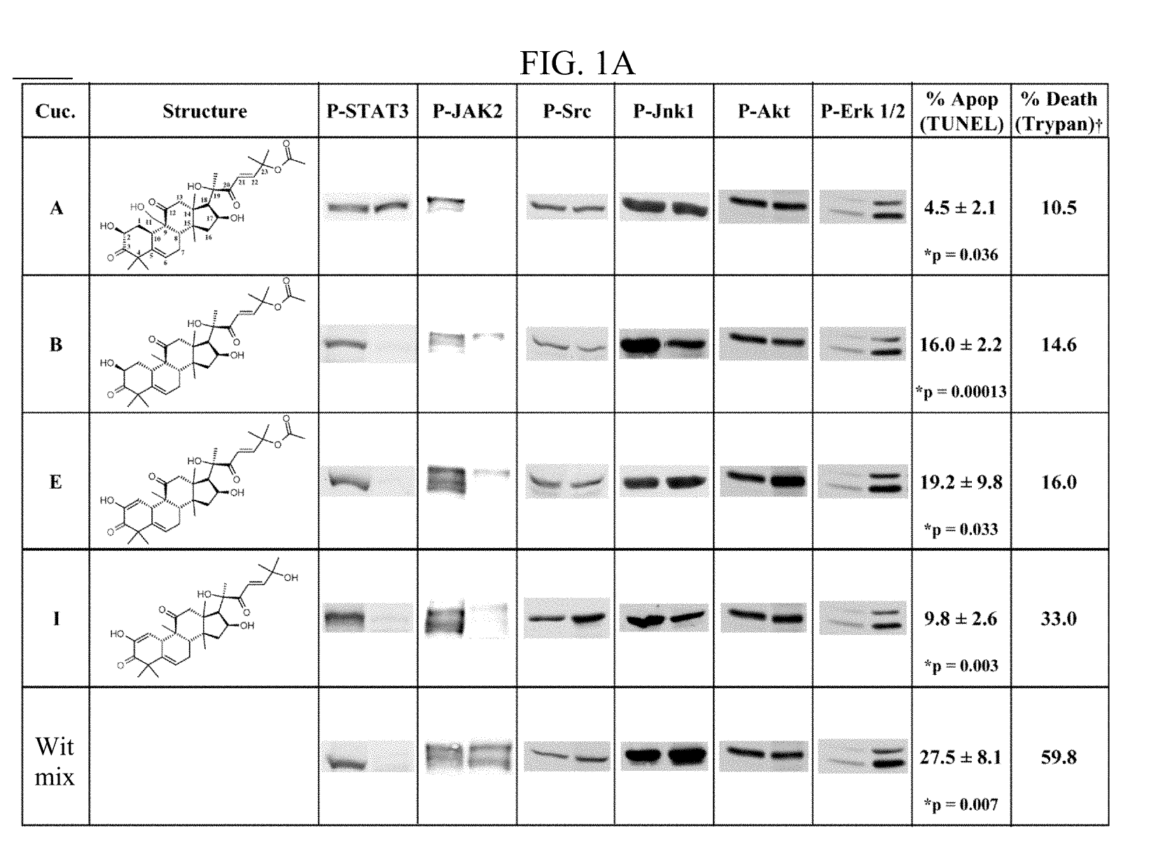

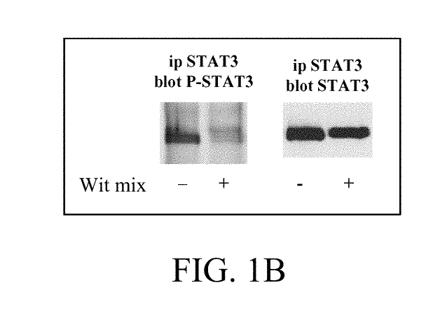

The identification of cucurbitacin I (JSI-124) as a potent inhibitor of activation of both JAK2 and STAT3 prompted the inventor to carry out SAR studies to identify agents that are selective for inhibiting the activation of either JAK2 or STAT3. To this end, A549 cells (a human non-small-cell lung carcinoma line) were treated with either vehicle, cucurbitacin analogs A, B, E, or I, or withacnistin mixture (10 μM) for 4 hours and the cell lysates processed for Western blotting with antiphosphotyrosine STAT3 (Y705) antibody or antiphosphotyrosine JAK2 (Y1007, Y1008) antibody as described under Materials and Methods. FIG. 1A shows that the withacnistin mixture suppressed the levels of P-STAT3 but had no effect on those of P-JAK2. In constrast, Cuc A suppressed the levels of P-JAK2 but had no effect on those of PSTAT3. Cuc B, E, and I inhibited both P-STAT3 and PJAK2 levels (FIG. 1A). The fact that Cuc B, E, and I, but not A, suppressed P-STAT3 levels in A549 cells indicates that addition of a single hydroxyl to carbon 11 of the cucurbitacin pharmacophore results in loss of anti-STAT3 activity (FIG. 1A; compare Cuc A to B). Similarly, the ability of Cuc A, B, E, and I to suppress P-JAK2 levels indicates that simple conversion of the carbon 3 carbonyl in the cucurbitacins to a hydroxyl results in loss of anti-JAK2 activity (FIG. 1A; compare withacnistin mixture to cucurbitacin B).

To confirm that the withacnistin mixture decreases phosphotyrosine levels of STAT3 without affecting total STAT3 levels, A549 cells were treated with either vehicle control or the withacnistin mixture (10 μM) for 4 hours, immunoprecipitated the lysates against whole STAT3, then blotted with both P-STAT3 and STAT3 antibodies as described under Materials and Methods. FIG. 1B shows that withacnistin treatment suppressed P-STAT3 without affecting total STAT3 levels. It was also shown that treatment of A549 cells with 10 μM Cuc I and A, like withacnistin, does not affect total STAT3 levels, and none of the three compounds affects total JAK2 levels (data not shown). As further support of the specific antiphosphotyrosine STAT3, but not antiphosphotyrosine JAK2, activity of withacnistin, A549 cells, as well as two breast carcinoma cell lines (MDA-MB-435 and MDA-MB-468) that also express constitutively activated JAK2 and STAT3, were treated with the withacnistin mixture at various concentrations, and determined IC₅₀ values of inhibition of STAT3 and JAK2 activation. Table 1 shows that in all three cell lines, withacnistin is a selective inhibitor of STAT3 activation over JAK2 activation, with IC₅₀ values of 3.7±1.7, 0.9±0.6, and 1.4±0.7 μM in A549, MDA-MB-435, and MDA-MB-468, respectively. In all three cell lines, JAK2 activation was not inhibited at withacnistin concentrations as high as 10 μM. Cuc A specifically inhibited JAK2 activation (IC₅₀s of 1.5±0.7, 0.65±0.05, and 0.86 μM for A549, MDA-MB-435, and MDA-MB-468, respectively) without affecting STAT3 activation at 10 μM. Cuc I inhibited the activation of both STAT3 and JAK2 but was more potent towards inhibiting JAK2 activation (Table 1). Thus, in all three cell lines, withacnistin (Wit) inhibits specifically STAT3 but not JAK2 activation and Cuc A inhibits JAK2 but not STAT3 activation whereas Cuc I inhibits the activation of both STAT3 and JAK2.

TABLE 1
IC₅₀ values of inhibition of phosphotyrosine-STAT3 and
phosphotyrosine-JAK2 in human tumor cell lines.

| | Wit | | Cuc I | | Cuc A | |
|---|---|---|---|---|---|---|
| Cell line | P-STAT3 | J-JAK2 | P-STAT3 | P-JAK2 | P-STAT3 | P-JAK2 |
| A549 | 3.7 ± 1.7 | >10 (n = 3) | 0.8 ± 0.7 | 0.25 ± 0.09 | >10 (n = 4) | 1.5 ± 0.7 |
| MDA-MB-435 | 0.9 ± 0.6 | >10 (n = 3) | 4.6 ± 1.9 | 0.18 ± 0.07 | >10 (n = 3) | 0.65 ± 0.05 |
| MDA-MB-468 | 1.4 ± 0.7 | >10 (n = 2) | 7.5 ± 1.5 | 0.40 ± 0.26 | >10 (n = 3) | 0.86, 0.86 |
| | | | | | | (n = 2) |

Data are representative of at least three independent experiments, unless otherwise indicated

### EXAMPLE 2

### Withacnistin and Cucurbitacins are Highly Selective for STAT3 and JAK2 Over Src, Akt, Erk, and JNK Signaling

It was next determined whether withacnistin and the Cuc analogs are selective for the JAK2/STAT3 pathway over other signal transduction pathways. To this end, A549 cells were treated with 10 μM of the different Cuc derivatives or the withacnistin mixture and processed the lysates for Western blotting with antibodies specific for phospho-Src, phospho-Erk1/2, phospho-JNK, and phospho-Akt as described under Materials and Methods. FIG. 1A shows that A549 cells possess constitutively phosphorylated Src, Erk1/Erk2, JNK1, and Akt in addition to phospho-STAT3 and phospho-JAK2. Treatment with the withacnistin mixture, for 4 hours at 10 μM significantly blocked STAT3 phosphorylation with little effect on phosphotyrosine levels of JAK2, Src, JNK1, or Akt. In contrast, Cuc A potently inhibited JAK2 phosphorylation, but showed little inhibitory activity against STAT3, Src, JNK1, and Akt. As noted above, the other Cuc compounds were able to inhibit both phosphotyrosine-STAT3 and phosphotyrosine-JAK2 but, like both withacnistin and Cuc A, these compounds showed little inhibitory effect on phosphotyrosine levels of Src, JNK1, and Akt. Interestingly, all of the Cuc analogs and withacnistin significantly increased the levels of phosphorylated Erk1/2 in A549 cells. Thus, these results demonstrate that cucurbitacins and withacnistin are highly selective for inhibition of the JAK/STAT3 pathway activation.

### EXAMPLE 3

### Inhibition of the Activation of JAK2, Src, JNK, Akt, and Erk is not Required for Induction of Apoptosis by Cucurbitacins and Withacnistin

The next objective was to determine whether the ability of the cucurbitacins and withacnistin to induce apoptosis is dependent on suppression of PJAK2 and/or P-STAT3 levels. To this end, A549 cells were treated with vehicle control, or cucurbitacins (10 μM), or the withacnistin mixture (10 μM) for 24 h, harvested the cells, and determined tumor cell death (trypan blue exclusion) and apoptosis (TUNEL) as described under Materials and methods. FIG. 1A shows that the most potent inducer of cell death and apoptosis was withacnistin (60 and 28%, respectively). The least potent was Cuc A (11 and 5%, respectively). Cuc B, E, and I also induced tumor cell death (15-33%) and apoptosis (10-19%). Taken together, the results of FIG. 1A demonstrate that decreasing P-JAK2 and increasing P-Erk1/2 levels are not sufficient for significant apoptosis induction, as indicated by the low potency of Cuc A. Furthermore, the results also demonstrate that decreasing the levels of P-JAK2, P-Src, P-JNK, and P-Akt is not required for induction of apoptosis as indicated by the high potency of withacnistin. Finally, the results also suggest that the ability of the cucurbitacins and withacnistin to induce apoptosis is related to their ability to suppress P-STAT3 but not P-JAK2 levels in A549 cells (compare withacnistin to A).

### EXAMPLE 4

### Induction of Apoptosis by Withacnistin is Selective for Cells that Express Constitutively Activated STAT3

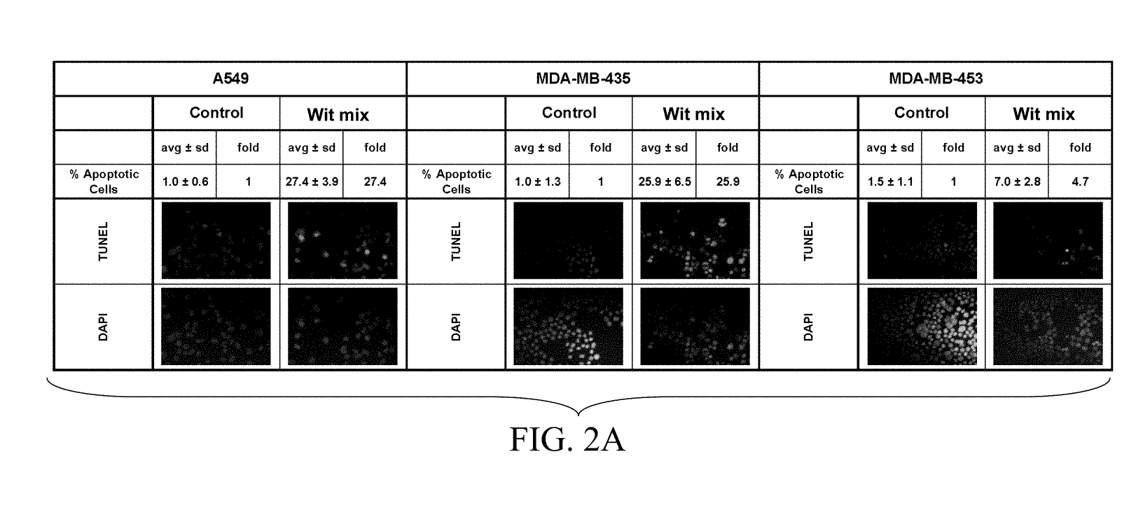

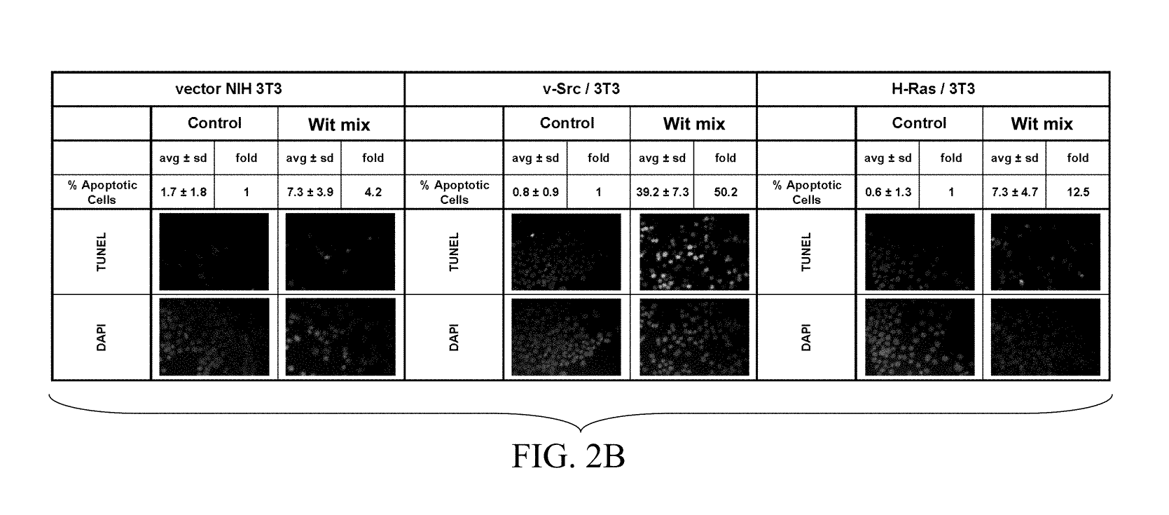

FIG. 1A SAR studies suggest that withacnistin induces apoptosis by blocking the activation of STAT3 in A549 cells. To give further support for this suggestion, it was next determined whether withacnistin induced apoptosis selectively in tumor cells that have high levels of activated STAT3 over those that do not. To this end, A549 cells and human breast carcinoma MDA-MB-435 cells which express very high levels of constitutively activated STAT3, and human breast carcinoma, MDA-MB-453, which do not show constitutive activation of STAT3 (Blaskovich, M. A. et al. Cancer Res., 2003, 63: 1270-1279; and data not shown), were treated for 24 hours with 10 μM withacnistin mixture or DMSO vehicle control. FIG. 2A shows that withacnistin only induced apoptosis strongly in the two cell lines expressing activated STAT3, but not in MDA-MB-453 cells. In A549 cells, withacnistin increased the percentage of apoptotic tumor cells by 27.4-fold compared to vehicle-treated control cells. In MDAMB-435 cells, withacnistin increased the percentage of apoptotic cells by a 25.9-fold. However, in MDA-MB-453 cells, withacnistin increased this percentage by only 4.7-fold (FIG. 2A).

To further confirm that tumor cells that depend on STAT3 for transformation are more sensitive to withacnistin-induced apoptosis compared to cell lines that do not depend on STAT3, v-Src/3T3 that contain constitutively-activated STAT3, oncogenic H-Ras/3T3, and vector-transfected NIH 3T3 cells that do not (Garcia, R. et al. Cell Growth Differ., 1997, 8: 1267-1276; Blaskovich, M. A. et al. Cancer Res., 2003, 63: 1270-1279) were treated with 10 μM withacnistin mixture for 24 hours. FIG. 2B illustrates the results from this experiment. A s with the human tumor cell lines, the v-Src/3T3 cell line, with its constitutively activated STAT3, showed a strong induction of apoptosis (from 0.8±0.9% in control compared to 39.2±7.3% with withacnistin treatment, a 50.2-fold increase). In contrast, the H-Ras/3T3 cell line showed significantly less induction of apoptosis (from 0.6±1.3% in control to only 7.3±4.7% with withacnistin treatment, a 12.5-fold increase). In vector/3T3 cells, withacnistin increased the percentage of apoptotic cells by only 4.2-fold (from 1.7±1.8% in control to 7.3±3.9% with withacnistin treatment) (FIG. 2B). Coupled with the human tumor cell results from FIG. 2A, these results demonstrate that withacnistin selectively induces more apoptosis in cell lines which express activated STAT3 compared to those with little or no STAT3 activation.

### EXAMPLE 5

### Withacnistin Inhibits A549 and v-Src Transformed NIH 3T3 Tumor Growth in Nude Mice

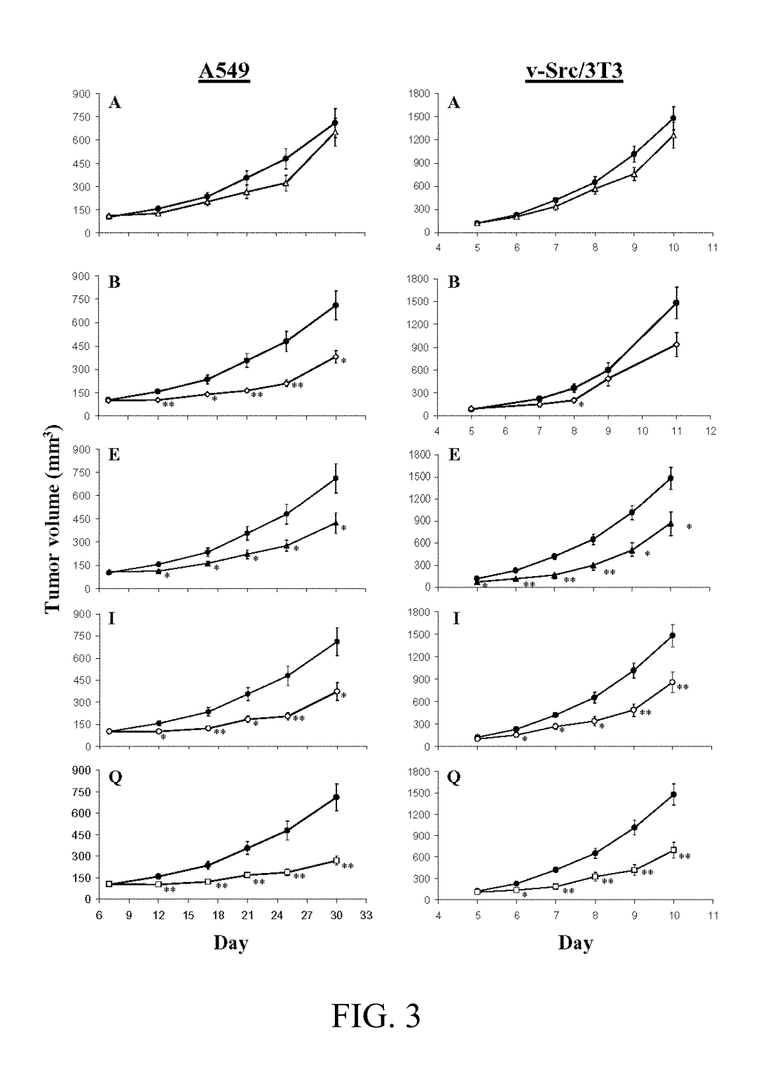

To determine the ability of the cucurbitacin analogs and withacnistin to inhibit tumor growth in vivo, the antitumor activity of the cucurbitacin analogs and withacnistin against both A549 and v-Src/3T3 tumors in a nude mouse xenograft model was evaluated. When the tumors became palpable (at volumes of approximately 100-150 mm³), the mice were treated either with vehicle control or 1 mg/kg/day of the cucurbitacins or the withacnistin mixture. Tumor volumes were monitored by caliper measurement as previously described (Blaskovich, M. A. et al. Cancer Res., 2003, 63: 1270-1279) and under Materials and Methods. FIG. 3 shows the antitumor efficacy of the cucurbitacin compounds and withacnistin. With A549 xenografts, all compounds except for Cuc A (11.1% inhibition, P=0.656) showed statistically significant inhibition of tumor growth. Withacnistin (wit) was highly potent, with 73.1% inhibition (P=0.001) of A549 tumor growth in nude mice (FIG. 3 and Table 2). Cuc I was a potent inhibitor of A549 tumor growth with 55.4% inhibition (P=0.011). Likewise, Cuc B (53.6% inhibition, P=0.010) and Cuc E (48.5%, P=0.024) were significant inhibitors of growth of A549 adenocarcinoma in nude mice (Table 2).

TABLE 2
Antitumor activity of cucurbitacin analogs

| | |
|---|---|
| v-Src/3T3 | A549 |

| Compound | % Inhibition | P^{a} | % Inhibition | P^{a} |
|---|---|---|---|---|
| Cuc A | 16 | 0.35 | 11.1 | 0.656 |
| Cuc B | 40^{a} | 0.006 | 53.6^{b} | 0.010 |
| Cuc E | 42 | 0.047 | 48.5 | 0.024 |
| Cuc I | 45 | 0.003 | 55.4 | 0.011 |
| Wit | 57 | 0.001 | 73.1 | 0.002 |

^{a}Two sided-Student's t-test.
^{b}Toxic at 1 mg/kg/day; results shown here are for 0.5 mg/kg/day.

In the v-Src/3T3 xenograft model, again Cuc A treatment did not result in statistically significant inhibition of tumor growth (16%, P=0.35). As in A549 tumors, withacnistin was highly potent at inhibiting the growth of v-Src/3T3 tumors. Withacnistin inhibited 57% of tumor growth while Cuc I, B, and E inhibited 45, 40, and 42% of tumor growth, respectively (FIG. 3 and Table 2). Taken together, and consistent with the in vitro data of FIG. 1A, the results of both xenograft models show that withacnistin is a potent and significant inhibitor of tumor growth, while Cuc A shows little ability to inhibit tumor growth in either model. Inhibition of STAT3 activity, with or without the ability to inhibit JAK2 activation (as with withacnistin and all cucurbitacins tested but Cuc A), results in antitumor activity, whereas inhibition of JAK2 activity, but not STAT3 activity (as with Cuc A), does not hinder the ability of the tumors to grow on nude mice. These results demonstrate that the ability of the withacnistin and the Cuc molecules to inhibit tumor growth is independent of their ability to inhibit JAK2 activation.

### EXAMPLE 6

### Immunohistochemical Analysis of Tumor Sections for STAT3 Activation and Apoptosis

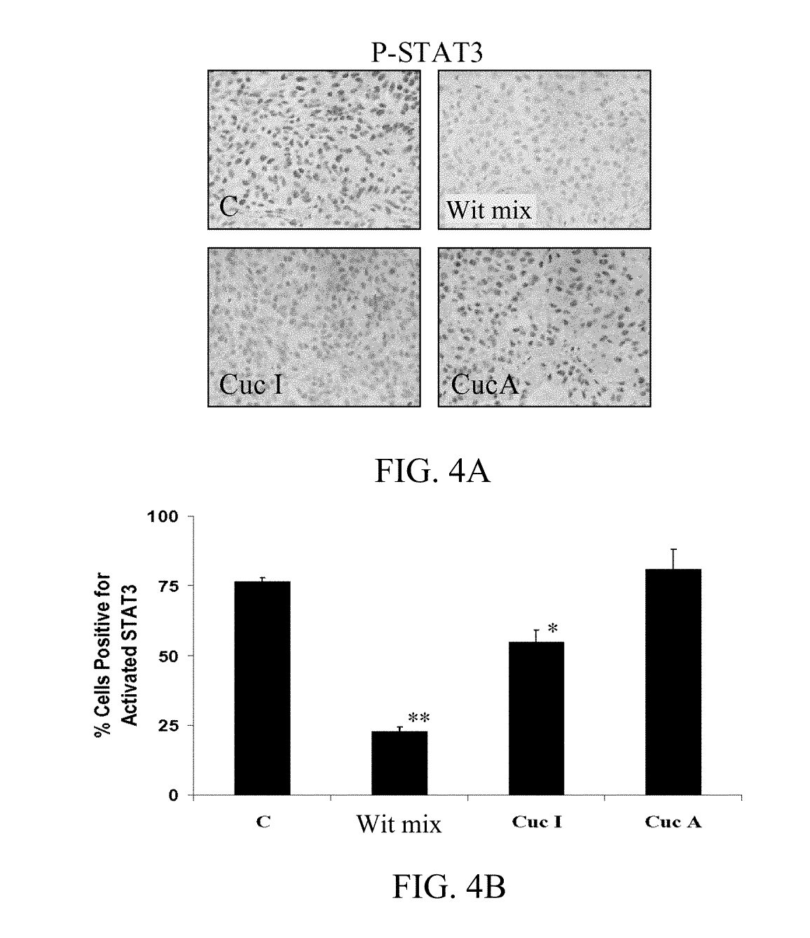

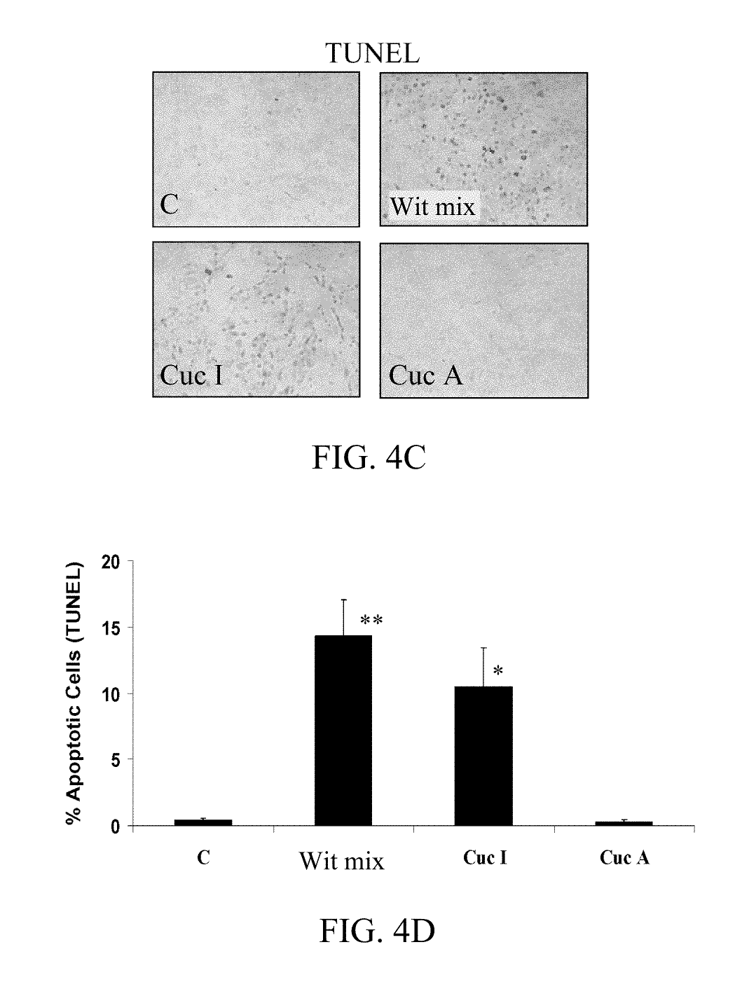

To determine whether phosphotyrosine STAT3 is targeted by withacnistin in vivo, and to determine if the results seen in cell culture concerning induction of apoptosis were occurring in tumors from animals treated with withacnistin, on the termination day of the A549 antitumor experiment, tumors from animals treated with Cuc A, Cuc I, and the withacnistin mixture, as well as vehicle control, were extracted and fixed in 10% neutral-buffered formalin and then processed into paraffin blocks for tissue sectioning. These tissue sections were stained separately with either TUNEL for determination of apoptosis, or phosphotyrosine STAT3 to determine if the signaling protein is inhibited in the tumors. Results of IHC staining are summarized in FIGS. 4A-4D. With P-STAT3 staining (FIG. 4A), it is apparent that both withacnistin and Cuc I inhibited STAT3 activation in A549 tumors, with withacnistin more potent than Cuc I (22.6±7.3% P-STAT3 positive cells for withacnistin and 54.7±4.5% for Cuc I compared to 76.5±1.4% for control; 70.5 and 28.5% inhibition of phosphotyrosine-STAT3 with withacnistin and Cuc I treatment, respectively), as shown in FIG. 4B. Cuc A showed virtually equal staining for phospho-STAT3 as vehicle control (80.8±1.8% P-STAT3-positive cells), indicating that there was no inhibition of STAT3 activation. TUNEL staining of tissue sections (FIG. 4C) revealed that, while Cuc A showed virtually no induction of TUNEL staining (0.3±0.2% TUNEL-positive cells) compared to control (0.4±0.1% TUNEL positive), both withacnistin (14.3±2.7%) and Cuc I (10.5±3.0%) showed strong staining for TUNEL, as shown in FIG. 4D, indicating the induction of apoptosis in the A549 cells comprising the tumors. As with the cell work, it is evident that only the two compounds that inhibit STAT3 activation demonstrate an ability to induce apoptosis.

Over the last decade overwhelming evidence has accumulated demonstrating the intimate involvement of STAT3 in malignant transformation and tumor survival. This prompted the development of inhibitors of STAT3 function as novel anticancer drugs. To this end, two approaches have been used, one targeting STAT3 dimerization (Turkson, J. et al. J. Biol. Chem., 2001, 276: 45443-45455; Turkson, J. et al. Mol. Cancer Ther., 2004, 3: 261-269), a step required for STAT3 activation and translocation to the nucleus; and the other, inhibition of the activation of STAT3 by reducing its cellular phosphotyrosine levels (Blaskovich, M. A. et al. Cancer Res., 2003, 63: 1270-1279). Recently, using a phosphotyrosine-STAT3 cytoblot to evaluate the NCI diversity set chemical library, Cuc I was identified, which inhibited both STAT3 and JAK2 activation (Blaskovich, M. A. et al. Cancer Res., 2003, 63: 1270-1279). In the present research, SAR studies with four cucurbitacin analogs and one compound previously misidentified as a cucurbitacin analog, led to the identification of a highly selective STAT3 activation inhibitor, withacnistin; a highly selective inhibitor of JAK2 activation, Cuc A; and three dual inhibitors, Cuc I, E, and B. From the chemical point of view, these are very important findings. For example, with respect to the cucurbitacins, these findings indicate that addition of a single hydroxyl group to carbon 11 of the cucurbitacins results in loss of anti-STAT3 activity, whereas a simple conversion of a carbon 3 carbonyl to a hydroxyl leads to loss of anti-JAK2 activity (see FIG. 1A).

Identifying compounds that are highly selective for either STAT3 or JAK2 allowed the investigation of important issues concerning the involvement of STAT3 versus JAK2 in human cancer cell survival. These studies suggest that suppressing STAT3 activation is more detrimental to tumor survival than blocking JAK2 activation. Indeed, both in cultured cells as well as in nude mouse xenografts, Cuc A, which blocks JAK2 but not STAT3 activation, was a poor inducer of apoptosis and an ineffective inhibitor of tumor growth. Furthermore, all three cucurbitacins (Cuc I, E, and B) that inhibit the activation of both STAT3 and JAK2 were less active at inducing apoptosis and inhibiting tumor growth suggesting that inhibition of JAK2 activation may hinder the antitumor activity of cucurbitacins.

Cancer is a result of many genetic alterations resulting in numerous aberrant signal transduction pathways (Hanahan, D. and Weinberg, R. A. Cell, 2000, 100: 57-70). Although activation of STAT3 is a major contributor to malignant transformation, other pathways such as those that mediate the action of the Ras and Src oncoproteins play pivotal roles in oncogenesis and tumor survival. An important question is whether suppression of all aberrant pathways is necessary for inducing tumor cell death. In these studies, it has been demonstrated that withacnistin, Cuc I, Cuc E, and Cuc B induced apoptosis without inhibiting the activation of Src, Akt, Erk1/2, and JNK, suggesting that the suppression of STAT3 activation is sufficient for apoptosis induction. This is consistent with the notion that many genetic alterations need to accumulate for cancer development and consequently suppressing one of these could be sufficient for reversal of malignant transformation.

The fact that withacnistin inhibits STAT3 activation whereas Cuc A inhibits JAK2 activation suggests that these compounds have different targets. The actual biochemical targets for cucurbitacins are not known. The lowering of phosphotyrosine levels suggest that these agents either inhibit upstream tyrosine kinases or activate upstream phosphotyrosine phosphatases. Possible tyrosine kinases that could be targets are the Src family of kinases. Src kinase itself was not inhibited in vitro by Cuc I (Blaskovich, M. A. et al. Cancer Res., 2003, 63: 1270-1279) and withacnistin (data not shown).

Withacnistin and Cuc A have distinct biological and physiological effects. Cuc A inhibited JAK2 but not STAT3 activation and was not able to induce apoptosis and inhibit tumor growth of the A549 lung tumors in nude mice. In contrast, withacnistin inhibited STAT3 but not JAK2 activation and was very potent at inducing apoptosis and at inhibiting A549 tumor growth in the same animal model. Furthermore, in cultured human cancer cells and oncogene-transformed murine cells, withacnistin induced programmed cell death much more efficiently in those tumors with constitutively activated STAT3. These SAR and in vitro/in vivo studies suggest that inactivation of JAK2 is not sufficient and that selective inhibition of STAT3 with pharmacological agents can lead to tumor cell death. This is consistent with previous studies that demonstrated that a dominant-negative form of STAT3 (STAT3-beta) can induce apoptosis in human cancer cells (Niu, G. et al. Cancer Res., 1999, 59: 5059-5063; Turkson, J. and Jove, R. Oncogene, 2000, 19: 6613-6626).

In conclusion, compounds described herein are highly selective for disrupting JAK2 or STAT3 signaling and can be used as chemical probes to dissect the importance of these signal transduction circuits in normal and pathophysiological conditions. The studies herein used these probes to demonstrate that disruption of STAT3, not JAK2, function is more detrimental to tumor survival. These results give further support for the use of STAT3 as a molecular therapeutic target to combat cancer.

### EXAMPLE 7

### Identification of NSC-135075 as a Mixture of Withacnistin, 3-methoxy-2,3-dihydrowithacnistin, and 3-ethoxy-2,3-dihydrowithacnistin

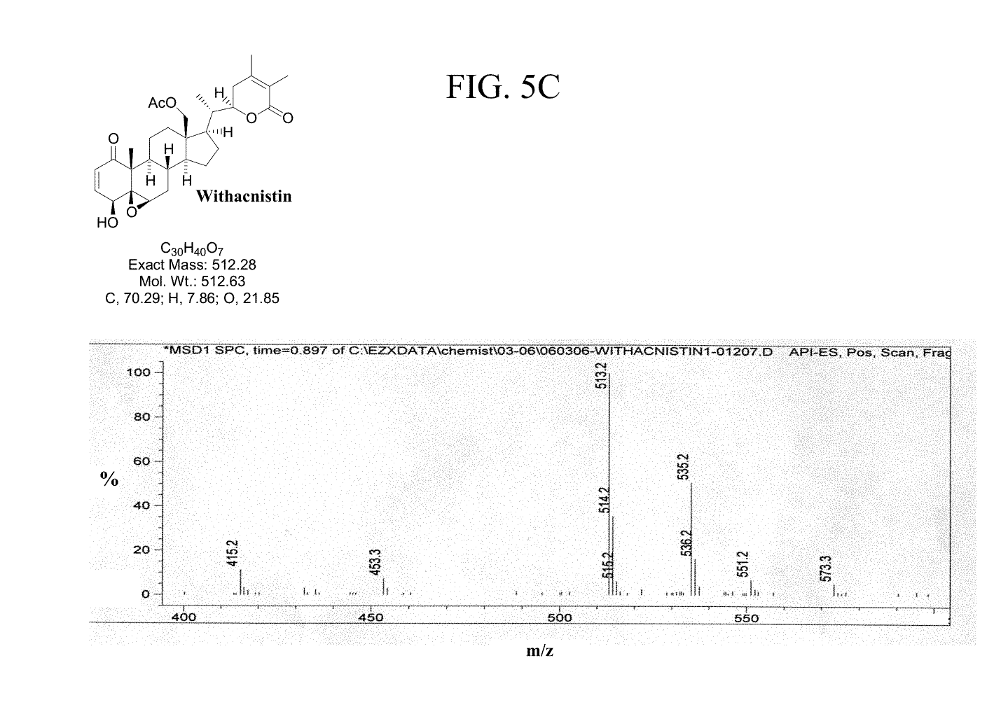

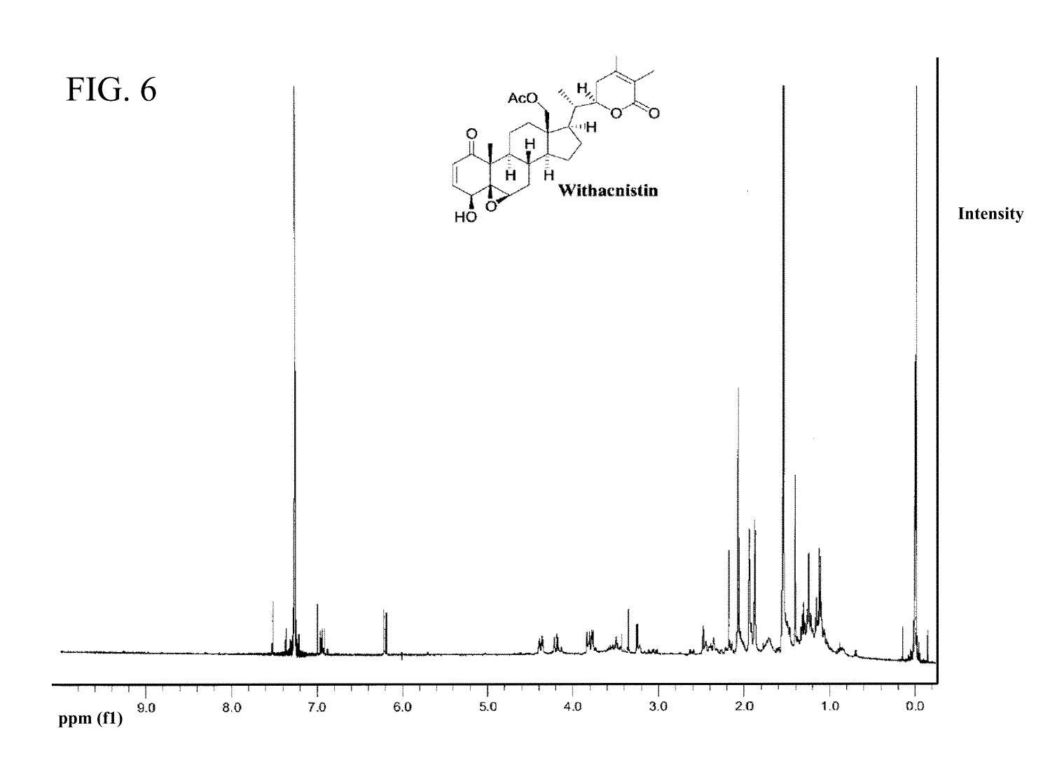

FIG. 6 shows an NMR spectrum of NSC-135075, showing peaks consistent with a withacnistin structure, instead of cucurbitacin Q. FIG. 5B shows an NMR spectrum of NSC-135075, the main peak showing that the sample is withcnistin. FIG. 5C shows the mass spectrum of the main pure peak of NSC-135075, showing the expected peak corresponding to M+H at m/z 513.

The H1 NMR of NSC-135075 is consistent with published NMR data of withacnistin. NMT data for withacnistin (2) from Alfonso, D. et al. (J. Nat. Prod., 1991, 54(6): 1576-1582).

TABLE 5
H-nmr Spectral Data of Relevant Protons of 1-3.^{a}

Compound

| Proton | 1 | 2 | 3 |
|---|---|---|---|
| H-2 | 6.21 (d, 10.1) | 6.20 (d, 10.1) | 6.21 (d, 10.1) |
| H-3 | 6.94 (dd, 6.1, | 6.94 (dd, .8, | 6.95 (dd, 58, |
| | 10.1) | 10.1) | 10.1) |
| H-4 | 3.77 (d, 6.1) | 3.77 (d, 5.8) | 3.77 (d, 5.8) |
| H-6 | 3.24 (br s) | 3.25 (br s) | 3.24 (br s) |
| H-7b | 2.16 (dt, 3.2, | 2.16 (dt, 2.9, | ^{b} |
| | 5.0, 14.9) | 4.8, 14.9) | |
| H-16 | — | — | 4.90 (t, 7.2, 9.0) |
| H-18 | 0.72 (s) | 3.83 (d, 11.9)^{c} | 0.76 (s) |
| | | 4.21 (d, 11.9) | |
| H-19 | 1.42 (s) | 1.41 (s) | 1.41 (s) |
| H-21 | 0.99 (d, 6.6) | 1.12 (d, 6.5) | 1.02 (d, 6.5) |
| H-22 | 4.43 (dt, 3.2, | 4.38 (dt, 3.2, | 4.17 (dt, 3.2, |
| | 4.3, 13.3) | 4.3, 13.3) | 4.3, 13.1) |
| H-23b | 2.50 (dd, 13.3, | 2.45 (br t) | 2.42 (br t) |
| | 18.0) | | |
| H-27 | 4.35 (d, 13.0)^{c} | 1.88 (s) | 1.88 (s) |
| | 4.39 (d)^{d} | | |
| H-28 | 2.04 (s) | 1.94 (s)^{e} | 1.93 (s)^{e} |
| H-30^{f} | — | 2.08 (s)^{e} | 1.96 (s)^{e} |

^{a}Chemical shifts are reported in ppm, signal multiplicities and coupling constants (Hz) are shown in parentheses.
^{b} Signal overlapped.
^{c}AB system.
^{d}J not measurable, the signal being overlapped by that of H-22.
^{e}Signals within a vertical column may be interchanged.
^{f}Protons from —OAc.

### EXAMPLE 8

### Withacnistin Inhibits P-STAT3 but not P-JAK2

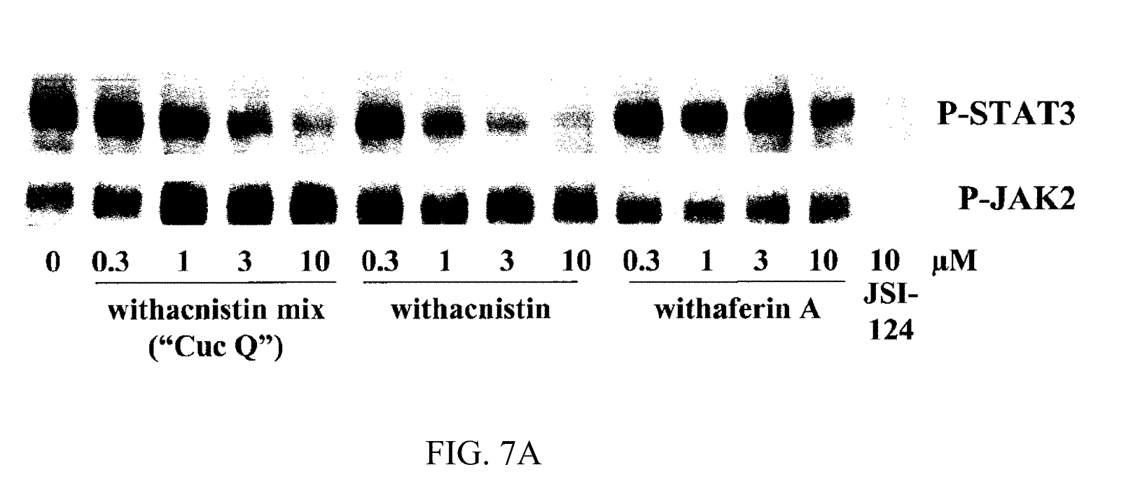

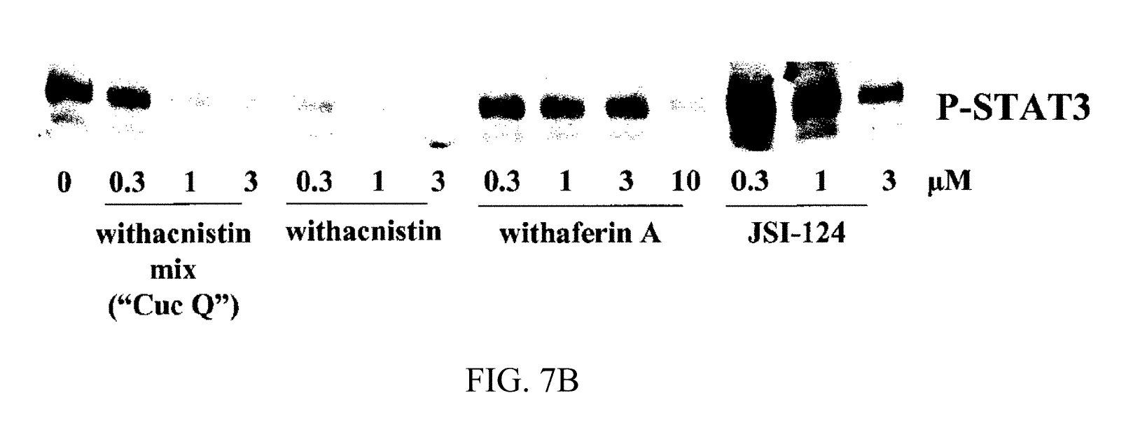

FIGS. 7A and 7B show that both the withacnistin mixture (mix) (a.k.a. NSC-135075), which was misidentified as cucurbitacin Q (CucQ), and pure withacnistin, inhibit P-STAT3 but not P-JAK2. Furthermore, pure withacnistin is more potent than the withacnistin mixture. FIG. 7A shows results from A549 cells following 4-hour treatment with withacnistin mix, pure withacnistin, withaferin A, or JSI-124. FIG. 7B shows results from MDA-MB468 cells following 4-hour treatment with withacnistin mix, pure withacnistin, withaferin A, or JSI-124.

### EXAMPLE 9

### NSC135075 is not Cucurbitacin Q but Rather a Mixture of Withacnistin, 3-methoxy-2,3-dihydrowithacnistin, and 3-ethoxy-2,3-dihydrowithacnistin

Previously, it was shown by the present inventor that treatment of human cancer cells that contain persistently activated hyper-phosphorylated STAT3 (P-STAT3) with the NCI library compound NSC135075 resulted in suppression of P-STAT3 levels and induction of apoptosis (Sun et al., Oncogene, 2005, 24: 3236-3245). According to NCI records, NSC135075 corresponds to cucurbitacin Q (Cuc Q) and, therefore, in the inventor's previous publication, it was referred to as such. However, recently NCI notified the present inventor that HPLC (FIG. 5A) and NMR (FIG. 5B) studies revealed that NSC135075 is composed of a mixture of a main peak corresponding to the natural product withacnistin and two minor peaks corresponding to 3-ethoxyx-2,3,-dihyrowithacnistin (EDH-Wit and 3-methoxy-2,3-dihydrowithacnistin (MDH-Wit). The NMR of the major peak of NSC135075 is consistent with the published NMR data of Wit (J. Nat Products, 1991, 64(12): 1576-8, which is incorporated herein by reference in its entirety). Furthermore, mass spectrometry analysis of the main pure peak of NSC135075 gave a mass corresponding to Wit, not Cuc Q (FIG. 5C), further confirming that the major component in NSC135075 is not Cuc Q, but rather Wit.

### EXAMPLE 10

### Withacnistin is the Active Component of the NSC-135075 Mixture and Suppresses P-STAT3 but not P-JAK2 Levels

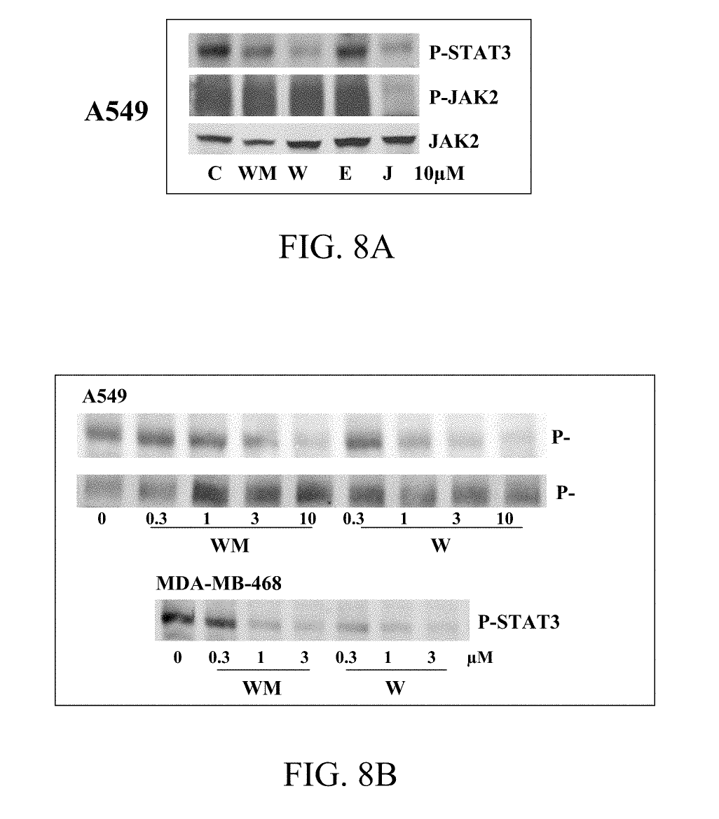

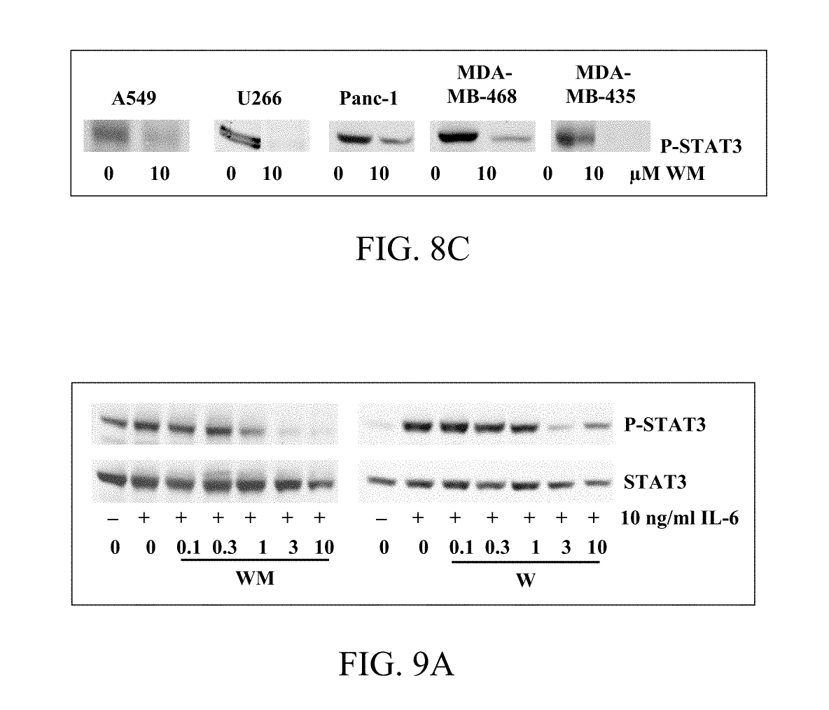

Having demonstrated that NSC-135075 is mainly composed of withacnistin (Wit) and 2 minor peaks, the inventor set out first to confirm that Wit suppresses P-STAT3 but not P-JAK2 as previously reported for the mixture that was thought to be Cuc Q. To this end, human lung cancer cells (A549) were treated with either the Wit mixture (Wit mix, or WM, NSC-135075), pure Wit or W, pure EDH-Wit, (no MDH-Wit was provided because NCI had none left) or JSI-124 (cucurbitacin I) a compound that has previously been shown to suppress both P-STAT3 and P-JAK2 (Blaskovich, M. A. et al. Cancer Res., 2003, 63: 1270-1279; Nefedova et al., J Immunol, 2005, 175(7): 4338-46). FIG. 8A shows that the WM and pure W suppressed P-STAT3 but not P-JAK2 levels. FIG. 8A also shows that EDH-Wit suppressed neither P-STAT3 nor P-JAK2 and that, as expected, JSI-124 suppressed the levels of both. FIG. 8B shows that in both A-549 cells as well as the MDA-MB-468 breast cancer cells W is slightly more potent than WM. These results demonstrate that the main component (W) of NSC-135075 is the active component. To determine if WM could suppress P-STAT3 levels in a variety of human tumors, in addition to A549, and MDA-MB-468, multiple myeloma (U266) cells, breast cancer MDA-MB-435 cells, and pancreatic cancer Panc-1 cells were treated, and found WM to be highly effective at suppressing P-STAT3 levels in all four cell lines.

### EXAMPLE 11

### Withacnistin Inhibits IL-6, IFN-b, EGF, and PDGF Stimulation of STAT3 but not STAT1 Tyrosine Phosphorylation in Human Cancer Cell Lines

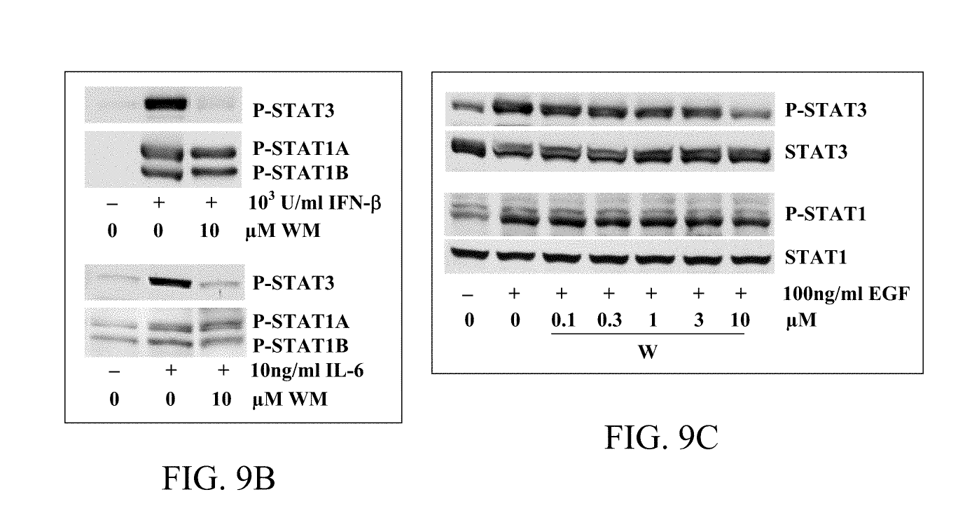

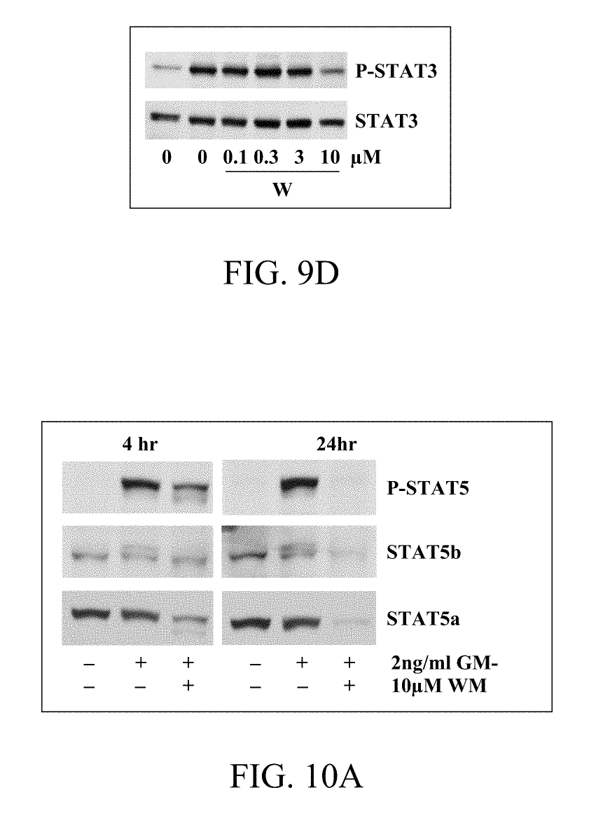

Previously, the inventor demonstrated that Wit mix suppresses the levels of P-STAT3 and induces apoptosis preferentially in human cancer cells that contain persistently hyperactivated STAT3. However, this was demonstrated on constitutively activated P-STAT3 and, therefore, whether Wit has any effects on growth factor or cytokine activation (tyrosine phosphorylation) of STAT3 is not known. Furthermore, it is not know whether Wit suppresses constitutive or stimulated P-STAT3 levels selectively over other STAT family members such as STAT1 and STAT5. To this end, a variety of human cancer cell lines were treated with Wit and stimulated with growth factors or cytokines known to activate STAT family members as described under Methods. FIG. 9A shows that treatment of the human multiple myeloma cell line U266 with interleukin-6 (IL-6) resulted in stimulation of STAT3 tyrosine phosphorylation. Pretreatment of U266 cells with W or WM blocked this IL-6 activation of STAT3 in a dose-dependent manner. In contrast, IL-6 activation of STAT1 in these cells was not affected by Wit pretreatment (FIG. 9B). FIG. 9B also shows that, similar to the results of FIGS. 9A and 9B, WM blocked the ability of interferon-beta (IFN-b) to stimulate tyrosine phosphorylation of STAT3 but not STAT1a or STAT1b in U266 cells. Similarly, FIG. 9C shows that W inhibited EGF activation of STAT3 but not STAT1 in breast cancer MDA-MB-468 cells. Finally, FIG. 9D shows that PDGF-stimulated tyrosine phosphorylation of STAT3 also is inhibited by pretreatment with W. Because the present inventor has minimal amounts of the purified Wit, most of the remaining experiments in this study had to be carried out with Wit mix.

### EXAMPLE 12

### Withacnistin Inhibits GM-CSF and PDGF Stimulation of STAT5 Tyrosine Phosphorylation

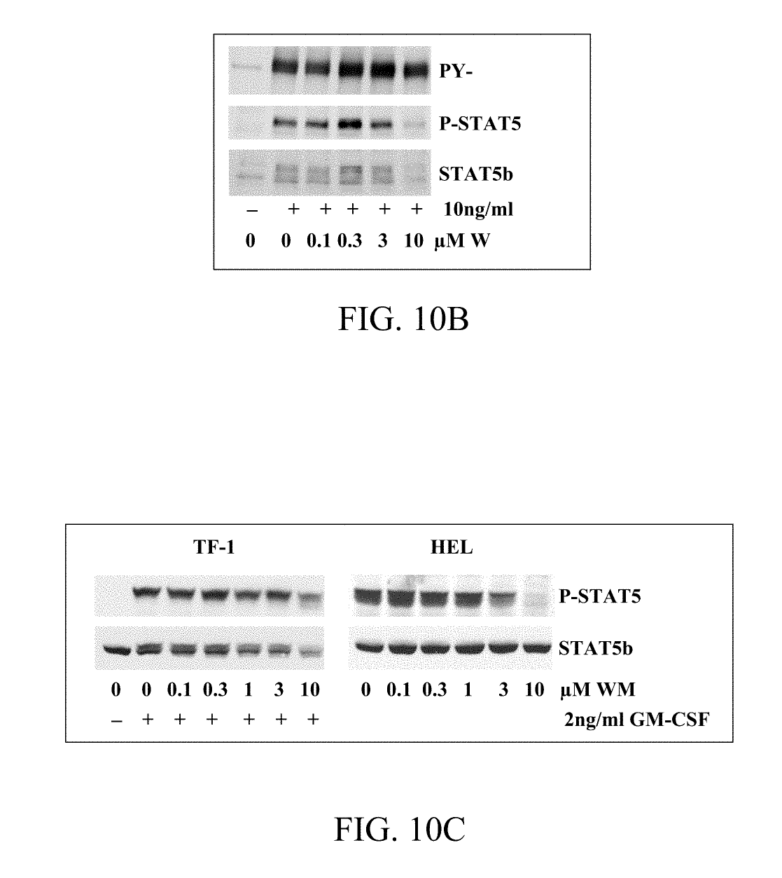

FIGS. 9A-9D clearly demonstrate that the ability of growth factors and cytokines to activate STAT3, but not STAT1, is hampered by the natural product withacnistin. The fact that Wit blocks STAT3 and not STAT1 activation in tumor cells is consistent with its ability to induce apoptosis in human cancer cells since STAT3 promotes, whereas STAT1 is believed to suppress, oncogenesis. To further establish the ability of Wit to suppress oncogenic signaling, its effects on cytokine stimulation of STAT5, another STAT family member known to promote oncogenesis, were determined. FIG. 10A shows that treatment of human erythroleukemia cells with GM-CSF for four or 24 hours resulted in a robust stimulation of the tyrosine phosphorylation of STAT5 and the pretreatment with WM inhibited this stimulation. Interestingly, WM also decreased the levels of STAT5a and STAT5b, especially at the 24 hour time point. FIGS. 10B and 10C show that WM inhibited GM-CSF stimulation of STAT5 in TF-1 cells as well as PDGF-stimulation of STAT5 in NIH 3T3 cells. It is important to note that WM did not inhibit PDGF stimulation of tyrosine phosphorylation of PDGF receptors in NIH 3T3 cells (FIG. 10B). Finally, constitutive levels of P-STAT5 in HEL cells were also inhibited by treatment with WM (FIG. 10C).

### EXAMPLE 13

### Withacnistin Induces the Levels of the STAT3 Negative Regulator SOCS3

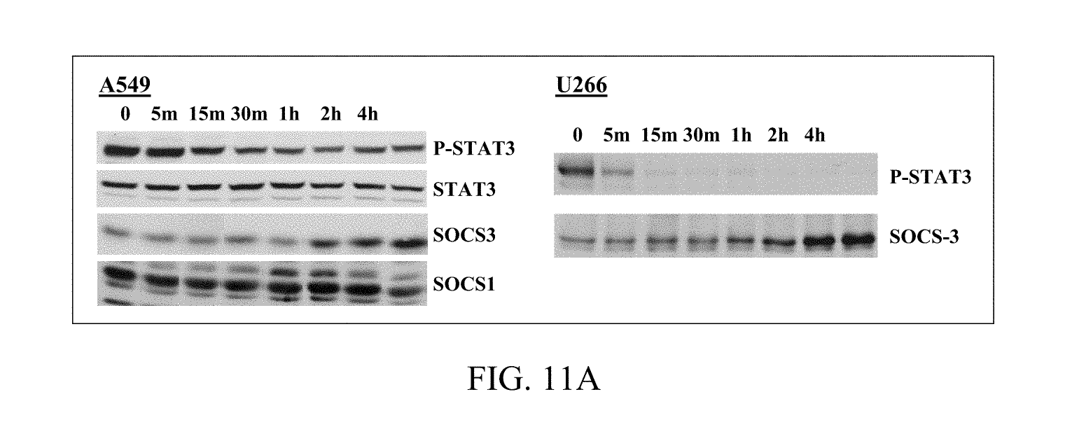

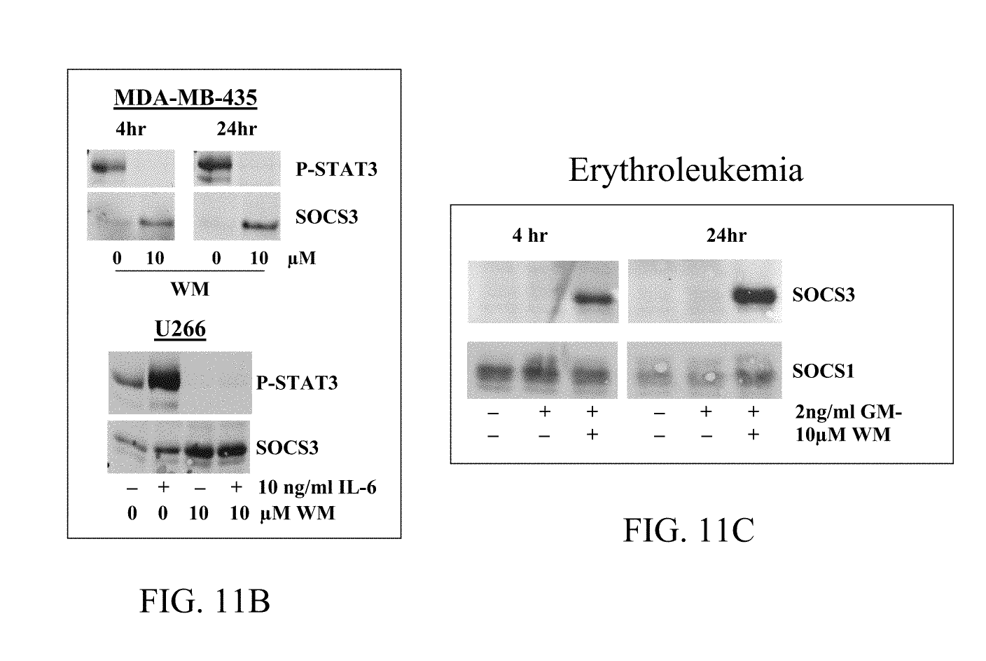

Activation of STAT3 occurs through its tyrosine phosphorylation. Inactivation of STAT3 can occur through several mechanisms including dephosphorylation by phosphotyrosine protein phosphatases as well as blocking STAT3 activation by SOCS3, which binds and prevents kinases from activating STAT3. To determine whether Wit also affects negative regulators of STAT3, its effects on SOCS3 were determined. To this end, A549 cells were first treated with Wit for various periods of time and its effects on both P-STAT3 and SOCS3 were determined. FIG. 11A shows that within 15 minutes treatment with WM, P-STAT3 levels begin to decrease without affecting total STAT3 levels for up to six hours. However, by 24 hours, Wit suppressed both P-STAT3 and total STAT3 levels (data not shown). FIG. 11A also shows that Wit induced the levels of SOCS3, but unlike the effects on P-STAT3, the induction of SOCS3 was detectable only after two hours of treatment. Similar results were also seen in U266 cells where WM inhibited P-STAT3 within 5 minutes and induced SOCS3 within 1 hour of treatment (FIG. 11A). WM also inhibited P-STAT3 and induced SOCS3 levels in the human breast cancer MDA-MB-435 cell line (FIG. 11B). Finally, WM was able to induce SOCS3 in GM-CSF stimulated erythroleukemia cells as well as IL-6-stimulated U266 cells (FIGS. 11A and 11C). It is important to note that WM had little effect on SOCS1 protein levels (FIGS. 11A and 11C).

Persistently hyperactivated, tyrosine phosphorylated STAT3 (P-STAT3) is prevalent in the majority of human tumor types and contributes greatly to malignancy and tumor survival. In this study, the present inventor identifies the natural product, withacnistin (Wit) as a STAT3 activation inhibitor and as an inducer of SOCS3, a negative regulator of STAT3. Inhibition of STAT3 activation occurs within 5 minutes, whereas induction of SOCS3 requires one hour. In a variety of human tumor cell lines, the ability of growth factors and cytokines, such as PDGF, EGF, IL-6, and IFN-β, to induce tyrosine phosphorylation of STAT3 is blocked by Wit. Furthermore, Wit also is able to block GM-CSF activation of STAT5. In contrast, the ability of IFN-β, IL-6, and EGF to activate STAT1 is not inhibited by Wit. Finally, in a variety of human cancer cell lines, Wit induces the levels of SOCS3, but not SOCS1. Together, these results identify Wit as a disruptor of STAT3- and STAT5-dependent oncogenic and tumor survival pathways in human cancer cells.

It should be understood that the examples and embodiments described herein are for illustrative purposes only and that various modifications or changes in light thereof will be suggested to persons skilled in the art and are to be included within the spirit and purview of this application.

What is claimed is:

1. A method for treating cancer in a patient, comprising administering withacnistin, or a pharmaceutically acceptable salt or analog thereof, to a patent in need of treatment.

2. A method for inhibiting the growth of cancer cells in a patient, comprising administering a pharmaceutical composition comprising a P-STAT inhibitor to the patient, the P-STAT inhibitor consisting essentially of withacnistin, resulting in inhibited cancer growth.

3. A method for treating cancer in a patient, comprising administering a pharmaceutical composition comprising only one withacnistin compound, wherein the withacnistin compound is withacnistin or a pharmaceutically acceptable salt thereof.
